(19) United States
(12) Patent Application Publication
(10) Pub. No.: US 2024/0315242 A1
(43) Pub. Date: Sep. 26, 2024

(54) Antibacterial Compound

(71) Applicant: LG Chem, Ltd., Seoul (KR)

(72) Inventors: Leehyeon Baek, Daejeon (KR); JI Seok Lee, Daejeon (KR); Hyungsam Choi, Daejeon (KR); Seonjung Jung, Daejeon (KR); Soonhee Kang, Daejeon (KR); Haesung Yun, Daejeon (KR); Yoon Hyung Hur, Daejeon (KR)

(73) Assignee: LG Chem, Ltd., Seoul (KR)

(21) Appl. No.: 18/576,595

(22) PCT Filed: Jun. 28, 2022
(86) PCT No.: PCT/KR2022/009225

§ 371 Date: Jan. 4, 2024

(30) Foreign Application Priority Data
Jul. 16, 2021 (KR) 10-2021-0093703
Jun. 27, 2022 (KR) 10-2022-0078380

(51) Int. Cl. A01N 37/44; A01P 1/00; C07C 219/06

(52) U.S. Cl. CPC A01N 37/44; A01P 1/00; C07C 219/06

(57) ABSTRACT

Provided is a novel compound, more particularly, a novel compound of Chemical Formula 1 exhibiting an excellent antibacterial property while having a polymerizable functional group to prepare an antibacterial polymer,

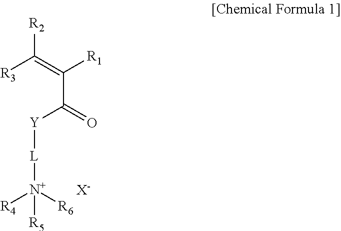

wherein all the variables are described herein.

### CROSS-REFERENCE TO RELATED APPLICATIONS

The present application is a national stage entry under 35 U.S.C. § 371 of International Application No. PCT/KR2022/009225 filed on Jun. 28, 2022, which claims priority from Korean Patent Application Nos. 10-2021-0093703, and 10-2022-0078380, filed on Jul. 16, 2021, and Jun. 27, 2022, respectively, all the disclosures of which are incorporated by reference herein in their entirety.

### TECHNICAL FIELD

The present disclosure relates to a novel compound exhibiting an antibacterial property.

### BACKGROUND ART

Recently, with the diversification of life, improvement of living standards, and change and improvement of consciousness, interest in improving hygiene and comfort in an individual's living environment is increasing. Accordingly, studies have been conducted on microorganisms that threaten them, but there are many types of microorganisms in the daily living environment, and they are widespread in nature, causing serious problems.

In particular, microorganisms such as bacteria, fungi, etc. may inhabit various environments, such as dietary life, residential environments, clothing, industrial products, etc. However, since bacteria may cause various inflammations or food poisoning, and fungi may produce not only odors, but also various skin diseases, respiratory diseases, allergies, atopic dermatitis, etc., they are problematic. In addition, microorganisms living on the surface of electronic products and household goods may cause deterioration of product performance.

Accordingly, in order to prevent human damage caused by these microorganisms, various antibacterial substances have been developed to inhibit the growth of microorganisms or to kill microorganisms.

Specifically, antibacterial agents previously developed may be broadly divided into inorganic antibacterial agents and organic antibacterial agents. Inorganic antibacterial agents are antibacterial agents containing a metal such as silver, copper, etc., and have an advantage of maintaining the antibacterial property even under high temperature conditions due to excellent thermal stability. However, there are problems in that they are expensive and there is a possibility of discoloration due to the metal ions contained after processing. In addition, organic antibacterial agents have advantages in that they are cheaper than inorganic antibacterial agents and exhibit excellent antibacterial effects even in a small amount. However, organic antibacterial agents have a problem in that antibacterial durability is poor because there is a possibility of leaching after being applied to articles.

Moreover, although organic antibacterial agent may secure stability of products in terms of inhibiting microbial growth and killing microorganisms, they are toxic to cause irritation to the user's skin.

For this reason, it has been discussed to introduce an antibacterial substance in the form of a polymer into an article in order to prevent a decrease in the antibacterial property and safety problems due to the leaching of the antibacterial substance. Accordingly, there is a need for an antibacterial monomer which has a polymerizable functional group to synthesize an antibacterial polymer while having excellent antibacterial property of the compound itself.

### PRIOR ART DOCUMENT

### Patent Document

(Patent Document 0001) Korean Patent No. 10-0601393

### DISCLOSURE

### Technical Problem

There is provided a novel compound having an excellent bacterial growth-inhibitory effect.

There is also provided an antibacterial agent including the compound.

### Technical Solution

To achieve the above objects,
there is provided a compound represented by the following Chemical Formula 1:

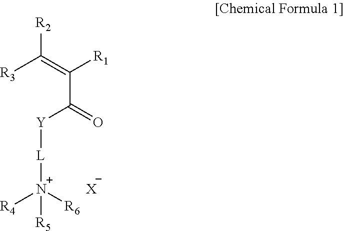

in Chemical Formula 1,
Y is O, S, or N(R₇),
L is a single bond, alkylene having 1 to 10 carbon atoms, or arylene having 6 to 60 carbon atoms,
R₁ to R₃ are each independently hydrogen or methyl,
R₄ to R₇ are each independently hydrogen, substituted or unsubstituted alkyl having 1 to 20 carbon atoms, or substituted or unsubstituted aryl having 6 to 60 carbon atoms, and
X⁻ is a conjugate base of an aromatic acid having 6 to 20 carbon atoms, which is substituted with one or more hydroxyl groups,
wherein the conjugate base of the aromatic acid may be further substituted with one or more substituents selected from the group consisting of halogen, alkyl having 1 to 4 carbon atoms, haloalkyl having 1 to 4 carbon atoms, and alkoxy having 1 to 4 carbon atoms.

There is also provided an antibacterial agent including the compound represented by Chemical Formula 1.

### Effect of the Invention

A compound according to the present disclosure is advantageous in that it has an excellent bacterial growth-inhibitory effect and has a polymerizable functional group to prepare a polymer exhibiting an antibacterial property.

### BRIEF DESCRIPTION OF DRAWINGS

FIG. 1 shows an MS spectrum of Compound A;

FIG. 2 shows an ¹H NMR spectrum of Compound A;

FIG. 3 shows an ¹H NMR spectrum of Compound 1; and

FIG. 4 shows a mass spectrum of Compound 1.

### Detailed Description

In the present invention, the terms “the first”, “the second”, and the like are used to describe a variety of components, and these terms are merely employed to differentiate a certain component from other components.

Further, the terms used in this description are just for explaining exemplary embodiments and it is not intended to restrict the present invention. The singular expression may include the plural expression unless it is differently expressed contextually. It must be understood that the term “include”, “equip”, or “have” in the present description is only used for designating the existence of characteristics taken effect, numbers, steps, components, or combinations thereof, and do not exclude the existence or the possibility of addition of one or more different characteristics, numbers, steps, components or combinations thereof beforehand.

Further, in the present invention, when a layer or an element is mentioned to be formed “on” or “above” layers or elements, it means that each layer or element is directly formed on the layers or elements, or other layers or elements may be formed between the layers, subjects, or substrates.

The present invention may be variously modified and have various forms, and specific exemplary embodiments are exemplified and explained in detail in the following description. However, it is not intended to limit the present invention to the specific exemplary embodiments and it must be understood that the present invention includes every modifications, equivalents, or replacements included in the spirit and technical scope of the present invention.

Further, the terms used in this description are just for explaining exemplary embodiments and it is not intended to restrict the present invention. The singular expression used herein may include the plural expression unless it is differently expressed contextually.

Meanwhile, as used herein, the term “(meth)acrylate” includes both acrylate and methacrylate.

In addition, in the present specification, the alkyl group may be straight or branched, and its number of carbon atoms is not particularly limited, but preferably 1 to 20. According to one embodiment, the number of carbon atoms of the alkyl group is 1 to 16. According to one embodiment, the number of carbon atoms of the alkyl group is 1 to 12. According to one embodiment, the number of carbon atoms of the alkyl group is 8 to 12. Specific examples of the alkyl group may include methyl, ethyl, propyl, n-propyl, isopropyl, butyl, n-butyl, isobutyl, tert-butyl, sec-butyl, 1-methyl-butyl, 1-ethylbutyl, pentyl, n-pentyl, isopentyl, neopentyl, tert-pentyl, 1-ethyl-propyl, 1,1-dimethylpropyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, 2-methylhexyl, 3-methylhexyl, 4-methylhexyl, 5-methylhexyl, octyl, n-octyl, tert-octyl, 1-methylheptyl, 2-ethylhexyl, 2,4,4-trimethyl-1-pentyl, 2,4,4-trimethyl-2-pentyl, 2-propylpentyl, n-nonyl, 2,2-dimethylheptyl, etc., but are not limited thereto. Further, in the present specification, the above description of the alkyl group may also be applied to alkylene, except that the alkylene is a divalent group.

Further, in the present specification, the aryl group is not particularly limited, but may preferably have 6 to 60 carbon atoms, and may be a monocyclic aryl group or a polycyclic aryl group. According to one embodiment, the number of carbon atoms of the aryl group is 6 to 20. According to one embodiment, the number of carbon atoms of the aryl group is 6 to 10. The monocyclic aryl group may include a phenyl group, a biphenyl group, a terphenyl group, etc., but is not limited thereto. The polycyclic aryl group may include a naphthyl group, an anthracenyl group, a phenanthryl group, a pyrenyl group, a perylenyl group, a chrysenyl group, a fluorenyl group, etc., but is not limited thereto. Further, in the present specification, the above description of the aryl group may also be applied to arylene, except that the arylene is a divalent group.

Further, in the present specification, the term “substituted or unsubstituted” may be understood as the meaning of “being substituted with one or more, for example, 1 to 5 substituents selected from the group consisting of deuterium, halogen, cyano, alkyl having 1 to 10 carbon atoms, alkoxy having 1 to 10 carbon atoms, and aryl having 6 to 20 carbon atoms or being unsubstituted.

Further, in the present specification, the alkoxy group is a substituent in which an oxygen atom is bound to a connection portion with another atom in the alkyl group, and the above description of the alkyl group may also be applied to the alkyl group.

In order to bring antibacterial property to household chemical products generally used in daily living space, such as homes, offices, multiuse facilities, etc., antibacterial coating capable of preventing and/or killing growth of microorganisms such as bacteria is performed on the surface of the household chemical products. At this time, antibacterial agents contained in the antibacterial coating damage the cell membrane or cell wall of microorganisms or induce denaturation of their proteins, thereby inhibiting the growth of microorganisms, leading to preventing reproduction of microorganisms and/or killing microorganisms.

Further, there are more than 5,000 species of bacteria that have been identified. Specifically, bacteria have diverse cell morphologies such as spheres, rods, spirals, etc., and their demand for oxygen is also different from each other, and thus they are divided into aerobic bacteria, facultative bacteria, and anaerobic bacteria. Therefore, it is usually not easy for one type of antibacterial agent to have physical/chemical mechanisms whereby cell membranes/cell walls of various bacteria are damaged or proteins thereof are denatured.

In addition, there have been a problem that the antibacterial agent used in the article was leached over time, and there has been a concern that a user's health may be threatened by the antibacterial substance, when the user is exposed to the leached antibacterial agent. Accordingly, it has been discussed to introduce the antibacterial agent in the form of a polymer rather than a single compound, in order to prevent degradation of antibacterial property and safety problems due to the leaching of the antibacterial substance.

Accordingly, the present inventors found that when a compound has a structure of a salt compound, in which a quaternary ammonium cation having a polymerizable functional group and an anion having a specific structure are bound, the compound itself may exhibit antibacterial property against at least one of Gram-positive bacteria or Gram-negative bacteria, more specifically, all of Gram-positive bacteria and Gram-negative bacteria, while enabling synthesis of an antibacterial polymer such as a homopolymer or a copolymer due to the polymerizable functional group introduced into the molecule, thereby completing the present disclosure.

In particular, the anion included in the compound is not a halogen anion, but a conjugate base of an aromatic acid having 6 to 20 carbon atoms, which is substituted with one or more hydroxyl groups (—OH). More specifically, the anion has an aromatic ring compound structure, in which one or more hydroxyl groups (—OH) and one or more carboxylate group (—COO—) are substituted. In the case of having such a structure, the hydroxyl group (—OH) contained in the molecule interacts with the surface of the bacterial cell to decompose the cell membrane and to coagulate the contents, thereby further improving the antibacterial property of the compound. In addition, a stable salt may be formed due to the carboxylate group (COO—) contained in the molecule.

In addition, as used herein, the meaning of “exhibiting an antibacterial property against a specific bacterium” is that a compound (bacteriostatic substance) to be tested whether or not it has an antibacterial property is added to a culture medium of the test bacterium, and after culturing, the number of bacteria is significantly reduced, as compared to a reference without the bacteriostatic substance, and specifically, it means that an antibacterial rate (%) calculated by the following Equation 1 according to evaluation of the antibacterial property to be described later is 70% or more.

$$Antibacterial rate ( \% ) = ( 1 - {A}_{s} / {A}_{0} ) \times 100 \qquad [ Equation 1 ]$$

in the equation,
Aₛ(Aₛₐₘₚₗₑ) represents absorbance of the culture medium containing the bacteriostatic substance at a wavelength of 600 nm, and
A₀(A_{reference}) represents absorbance of the pure culture medium without the bacteriostatic substance at a wavelength of 600 nm.

More preferably, “exhibiting an antibacterial property against a specific bacterium” means that the antibacterial rate (%) calculated according to Equation 1 is 70% or more, 70.6% or more, 75.5% or more, 80% or more, 90% or more, 95% or more, 95.3% or more, 95.8% or more, 96% or more, 97% or more, 97.3% or more, 98% or more, 98.1% or more, and 100% or less.

Further, the Gram-positive bacteria collectively refer to bacteria that are stained purple when stained by the Gram staining method. The cell wall of Gram-positive bacteria consists of several layers of peptidoglycan. After staining with a basic dye such as crystal violet, even when the Gram-positive bacteria are treated with ethanol, they are not decolorized and remain colored purple. Bacteria categorized as the Gram-positive bacteria include Enterococcus faecalis, Staphylococcus aureus, Streptococcus pneumoniae, Enterococcus faecium, Lactobacillus lactis, etc.

Further, the Gram-negative bacteria collectively refer to bacteria that are stained red when stained by the Gram staining method, and have outer membrane consisting of lipopolysaccharides, lipoproteins, and other complex polymer materials instead of the cell wall having a relatively small amount of peptidoglycan, as compared to the Gram-positive bacteria. Thus, after staining with a basic dye such as crystal violet, even when the Gram-negative bacteria are treated with ethanol, they are decolorized, and when they are counter-stained with a red dye such as safranin, they appear red. Bacteria categorized as the Gram-negative bacteria include Proteus mirabilis, Escherichia coli, Salmonella typhi, Pseudomonas aeruginosa, Vibrio cholerae, etc.

Therefore, since the Gram-positive bacteria and Gram-negative bacteria may cause various diseases upon contacting, and may also cause secondary infection in critically ill patients with weak immunity, it is preferable that a single antibacterial agent is used to exhibit the antibacterial property against both Gram-positive bacteria and Gram-negative bacteria.

Meanwhile, the compound according to one embodiment may exhibit the antibacterial property against at least one of Gram-positive bacteria or Gram-negative bacteria due to the cation and the anion of the quaternary ammonium salt. Specifically, the ammonium cation of the quaternary ammonium salt is electrostatically adsorbed onto the cell wall of Gram-positive bacteria or Gram-negative bacteria, and then interaction with the alkyl group of the hydrophobic quaternary ammonium salt occurs, and as a result, the cell surface structure of the bacteria is destroyed to inhibit growth of the bacteria. Furthermore, the hydroxyl group (—OH) of the anion of the quaternary ammonium salt interacts with the surface of the bacterial cell to decompose the cell membrane and coagulate the contents, thereby further inhibiting growth of the bacteria.

Hereinafter, a compound and an antibacterial agent including the same will be described in more detail according to specific embodiments of the present invention.

### Compound

A compound of one embodiment is represented by the following Chemical Formula 1:

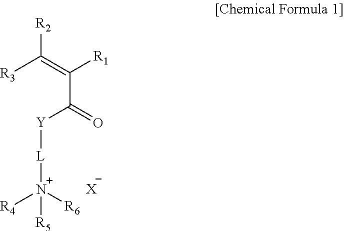

in Chemical Formula 1,
Y is O, S, or N(R₇),
L is a single bond, alkylene having 1 to 10 carbon atoms, or arylene having 6 to 60 carbon atoms,
R₁ to R₃ are each independently hydrogen or methyl,
R₄ to R₇ are each independently hydrogen, substituted or unsubstituted alkyl having 1 to 20 carbon atoms, or substituted or unsubstituted aryl having 6 to 60 carbon atoms, and
X⁻ is a conjugate base of an aromatic acid having 6 to 20 carbon atoms, which is substituted with one or more hydroxyl groups,
wherein the conjugate base of the aromatic acid may be unsubstituted or substituted with one or more substituents selected from the group consisting of halogen, alkyl having 1 to 4 carbon atoms, haloalkyl having 1 to 4 carbon atoms, and alkoxy having 1 to 4 carbon atoms, in addition to one or more hydroxyl groups.

Here, the conjugate base of the aromatic acid may be further substituted with one or more substituents selected from the group consisting of halogen, alkyl having 1 to 4 carbon atoms, haloalkyl having 1 to 4 carbon atoms, and alkoxy having 1 to 4 carbon atoms.

In other words, the conjugate base of the aromatic acid may be unsubstituted or substituted with one or more substituents, for example, 1 to 5 substituents selected from the group consisting of halogen, alkyl having 1 to 4 carbon atoms, haloalkyl having 1 to 4 carbon atoms, and alkoxy having 1 to 4 carbon atoms, in addition to one or more hydroxyl groups.

In Chemical Formula 1, Y may be O, S, or NH. Preferably, Y may be O.

In Chemical Formula 1, L may be methylene, ethylene or propylene.

Further, R₁ may be hydrogen or methyl, and R₂ and R₃ may be hydrogen.

Further, one of the three terminal groups, R₄, R₅ and R₆ substituents substituted in the quaternary ammonium cation of the first repeating unit may be alkyl having 5 to 20 carbon atoms. More specifically, one of R₄ to R₆ may be a linear, i.e., straight chain alkyl having 5 to 20 carbon atoms. In this regard, when all of R₄, R₅ and R₆ substituents are alkyl having less than 5 carbon atoms, there is a problem in that the antibacterial property may not be exhibited, and when any one of R₄, R₅ and R₆ substituents is alkyl having more than 20 carbon atoms, the starting material for preparing the copolymer is not dissolved in the solvent, and thus the synthesis itself is impossible.

More specifically, one of R₄ to R₆ may be alkyl having 5 to 20 carbon atoms, and the others may be each independently alkyl having 1 to 4 carbon atoms.

For example, one of R₄ to R₆ may be alkyl having 5 to 20 carbon atoms, and the others may be each independently methyl or ethyl. Preferably, R₅ may be alkyl having 5 to 20 carbon atoms, and R₄ and R₆ may be each independently methyl or ethyl.

More specifically, R₁ may be methyl, R₂ and R₃ may be hydrogen, one of R₄ to R₆ may be alkyl having 5 to 20 carbon atoms, and the others may be each independently methyl or ethyl; or
all of R₁ to R₃ may be hydrogen, one of R₄ to R₆ may be alkyl having 5 to 20 carbon atoms, and the others may be each independently methyl or ethyl.

Further, for example, one of R₄ to R₆ may be alkyl having 6 to 16 carbon atoms, and the others may be each independently methyl or ethyl. Preferably, R₅ may be alkyl having 6 to 16 carbon atoms, and R₄ and R₆ may be each independently methyl or ethyl.

Further, for example, one of R₄ to R₆ may be alkyl having 8 to 12 carbon atoms, and the others may be each independently methyl or ethyl. Preferably, R₅ may be alkyl having 8 to 12 carbon atoms, and R₄ and R₆ may be each independently methyl or ethyl.

In addition, among R₄, R₅ and R₆ substituents, two substituents other than alkyl having 5 to 20 carbon atoms may be the same as each other.

Preferably, in Chemical Formula 1, one of R₄, R₅ and R₆ may have 6 or more, 7 or more, or 8 or more carbon atoms, and 20 or less, 18 or less, 16 or less, 14 or less, or 12 or less carbon atoms.

For example, R₁ may be methyl, R₂ and R₃ may be hydrogen, R₄ may be alkyl having 6 to 16 carbon atoms, and R₅ and R₆ may be each independently methyl or ethyl. The antibacterial copolymer including the first repeating unit of the structure may exhibit excellent antibacterial property against at least one of Gram-positive bacteria or Gram-negative bacteria, more specifically, all of Gram-positive bacteria and Gram-negative bacteria.

Further, R₇ may be hydrogen, alkyl having 1 to 4 carbon atoms, or aryl having 6 to 20 carbon atoms. For example, R₇ may be hydrogen, methyl, or phenyl.

Meanwhile, in Chemical Formula 1, the compound includes a conjugate base of an aromatic acid having 6 to 20 carbon atoms, which is substituted with one or more hydroxyl groups (—OH), more specifically, an aromatic acid having 6 to 20 carbon atoms, which is substituted with 1 to 3 hydroxyl groups as a counter ion of the quaternary ammonium cation moiety.

As used herein, the “aromatic acid” is a kind of aromatic compound containing both an aromatic ring and an organic acid functional group, and specifically, refers to a compound in which one or more carboxyl groups (—COOH) are substituted on an aromatic ring having 6 to 20 carbon atoms. Therefore, the “conjugate base of an aromatic acid” may mean a compound in which one or more carboxylate groups (—COO⁻) are substituted on an aromatic ring having 6 to 20 carbon atoms, wherein a hydrogen ion (H⁺) is donated from the carboxyl group.

In other words, X⁻ is an aromatic ring compound having 6 to 20 carbon atoms, in which one or more hydroxyl groups and one or more carboxylate groups are substituted. When X⁻ is such a compound, the anion substitution reaction is advantageous, and thus preparation may be easy, and the antibacterial activity of the compound may also be further improved.

In this regard, the conjugate base of an aromatic acid may be further substituted with one or more, more specifically, 1 to 3 substituents selected from the group consisting of halogen, alkyl having 1 to 4 carbon atoms, haloalkyl having 1 to 4 carbon atoms, and alkoxy having 1 to 4 carbon atoms, in addition to the hydroxyl groups.

For example, X⁻ may be represented by the following Chemical Formula 2:

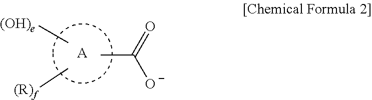

in Chemical Formula 2,
A is a benzene ring, or a naphthalene ring,
R is fluoro, bromo, chloro, iodo, methyl, ethyl, propyl, isopropyl, butyl, isobutyl, sec-butyl, tert-butyl, trifluoromethyl, methoxy, or ethoxy,
e is 1, 2, or 3, and
f is an integer of 0 to 5,
wherein when f is 2 or more, two or more R's are the same as or different from each other.

Specifically, in Chemical Formula 2,
R may be fluoro, bromo, chloro, methyl, trifluoromethyl, or methoxy,
e may be 1, 2, or 3, and
f may be 0, 1, 2, or 3.

At this time, e+f may be 1, 2, or 3.

More specifically, X⁻ may be represented by any one of the following Chemical Formulae 2-1 to 2-6:

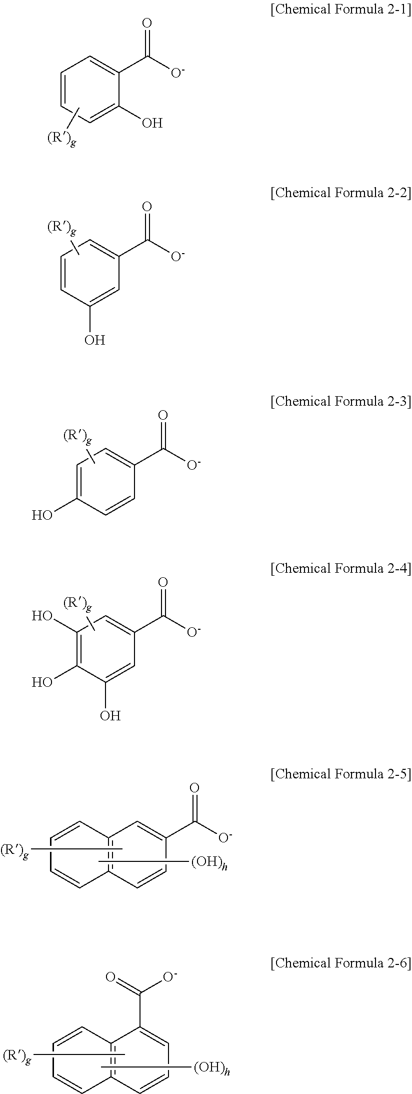

in Chemical Formulae 2-1 to 2-6,
R′ is fluoro, bromo, chloro, methyl, trifluoromethyl, or methoxy,
g is 0, 1, or 2, and
wherein when g is 2, two R's are the same as or different from each other,
h is 1 or 2.

At this time, g+h may be 1, 2, or 3.

For example, X⁻ is any one selected from the group consisting of:

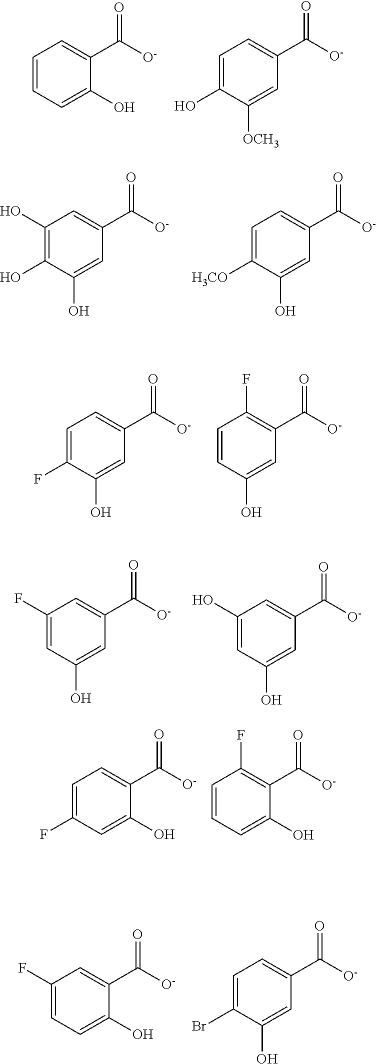

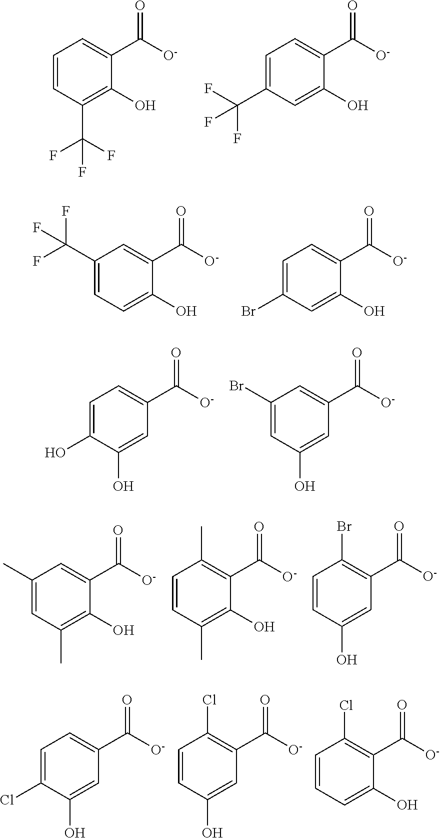

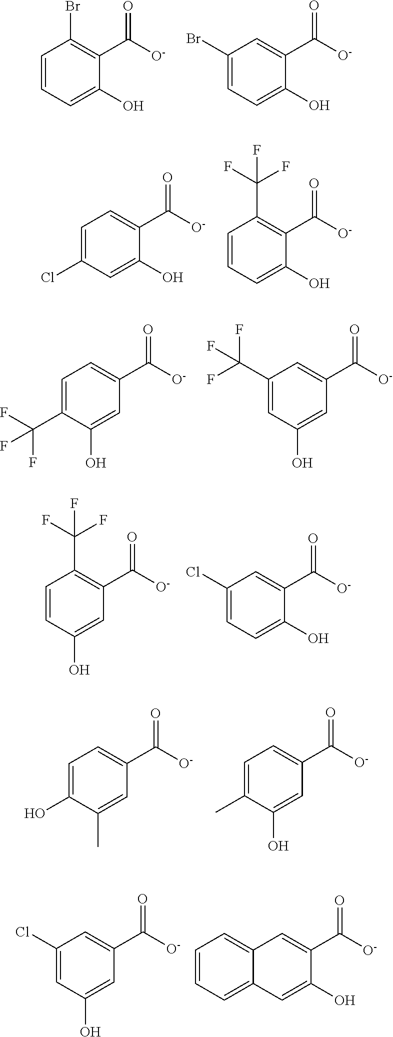

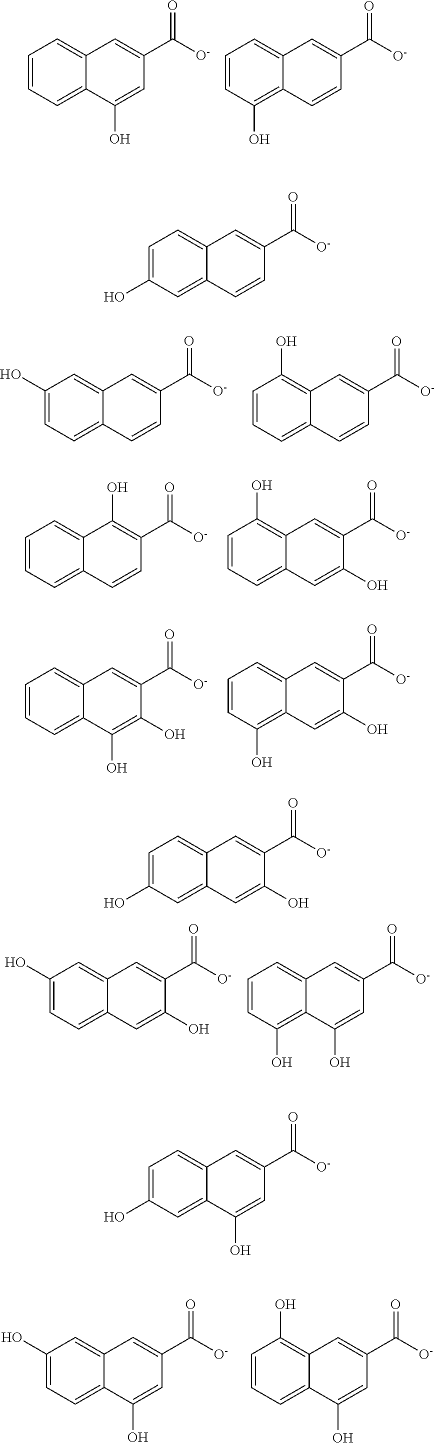

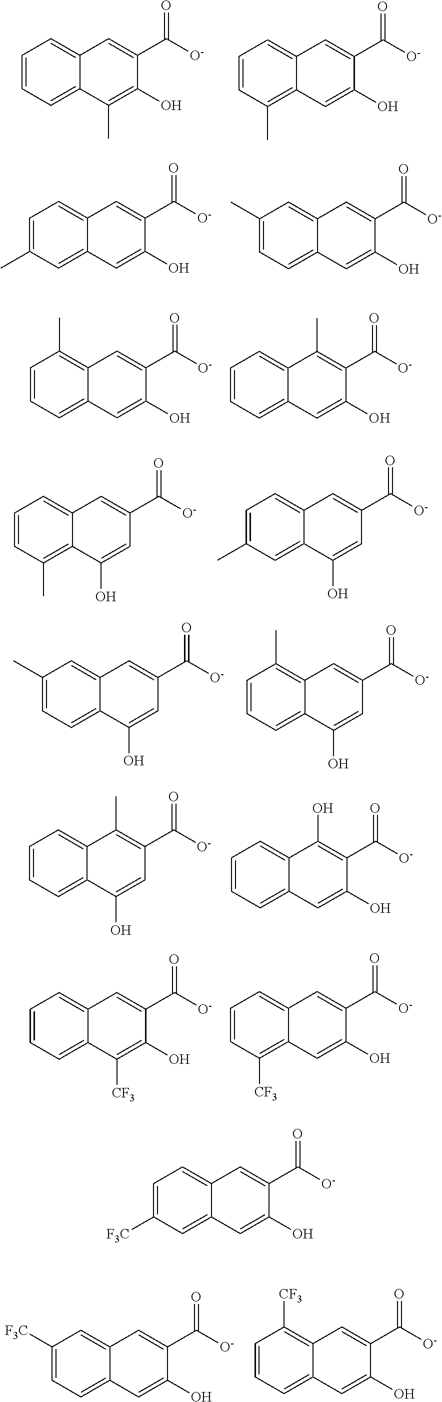

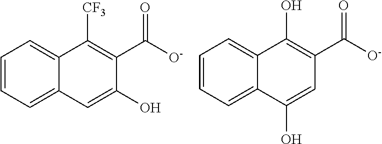 [SMILES: O=C([O-])c1c(O)cc2ccccc2c1C(F)(F)F ; O=C([O-])c1cc(O)c2ccccc2c1O]

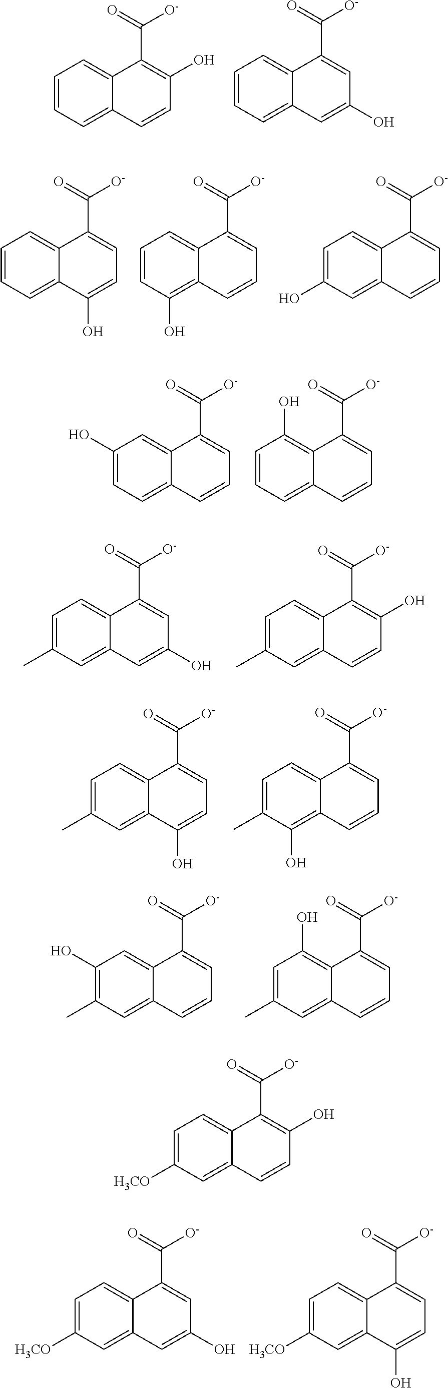

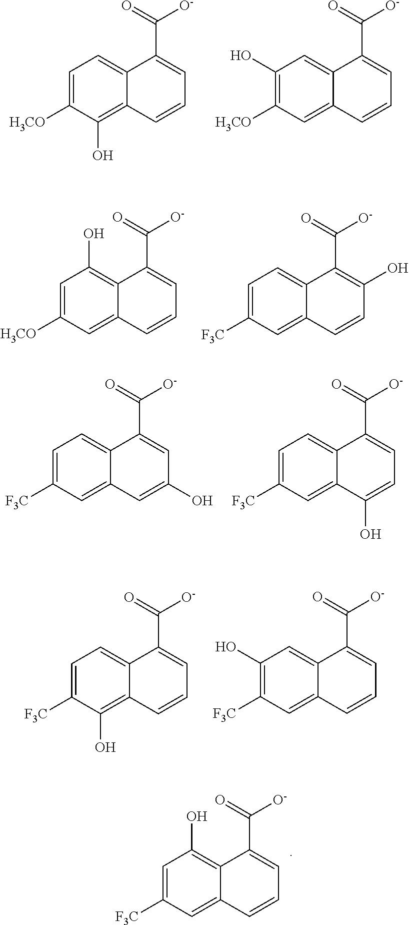

For example, the compound represented by Chemical Formula 1 may be represented by any one of the following Chemical Formulae 1-1 to 1-4:

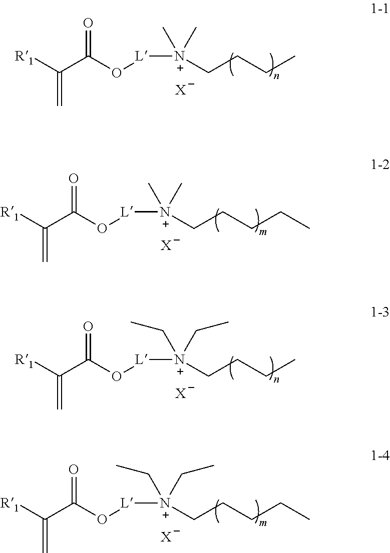

in Chemical Formulae 1-1 to 1-4,
R′₁ may be hydrogen or methyl,
L′ may be methylene, or ethylene,
X⁻ is the same as defined in Chemical Formula 1,
n is an integer of 2 to 7, and
m is an integer of 2 to 6.

In other words, in Chemical Formulae 1-1 to 1-4,
n may be 2, 3, 4, 5, 6, or 7, and
m may be 2, 3, 4, 5, or 6.

Meanwhile, the compound is any one selected from the group consisting of the following compounds:

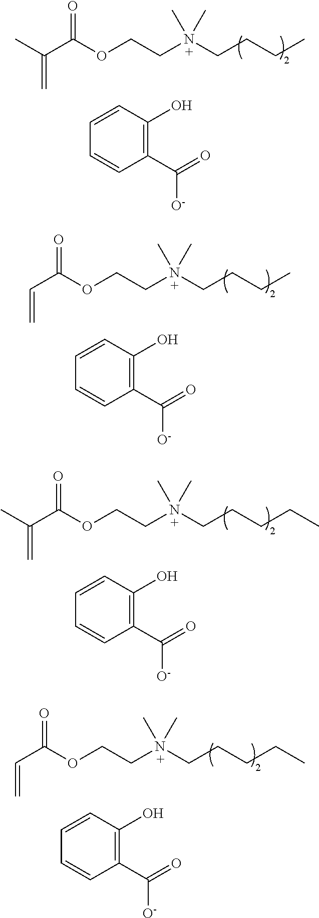

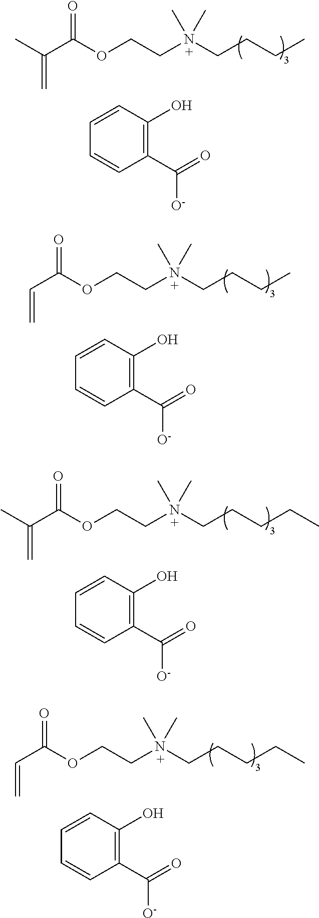

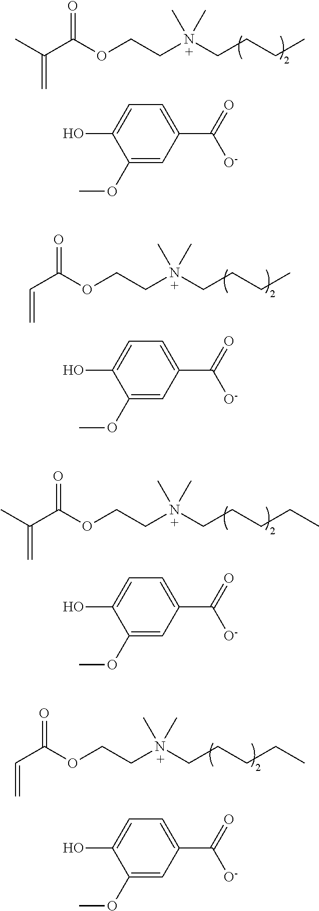

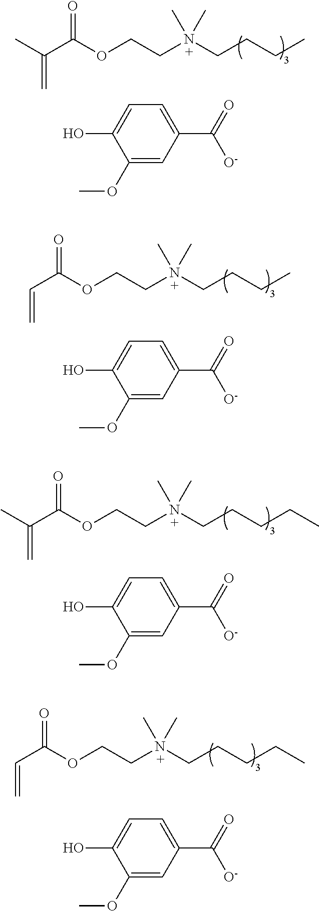

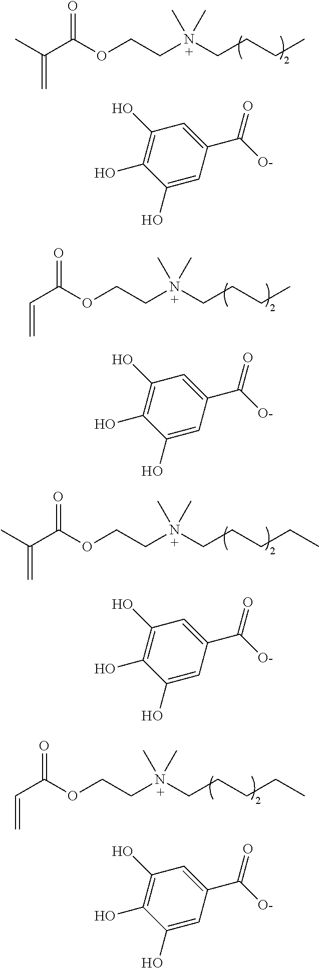

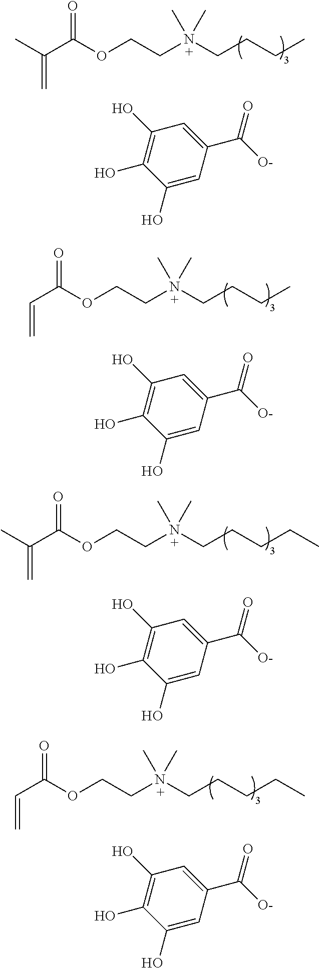

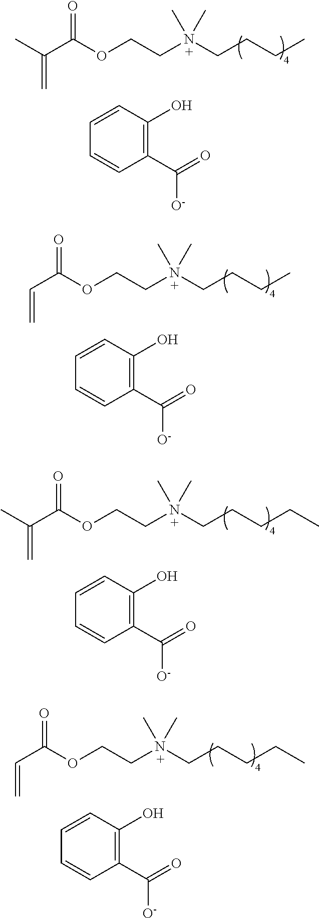

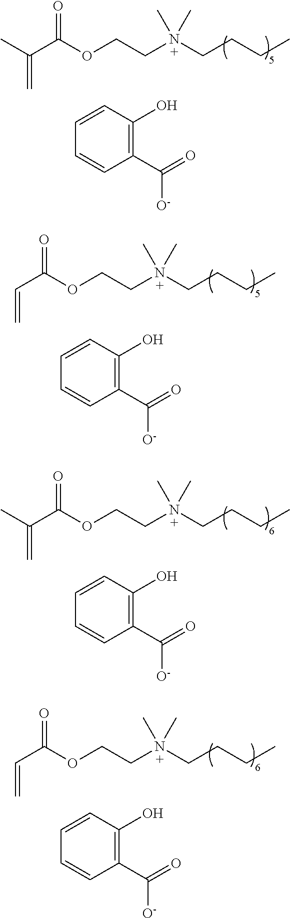

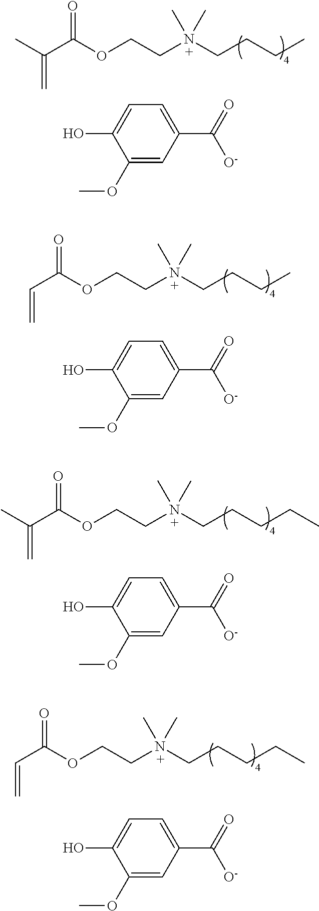

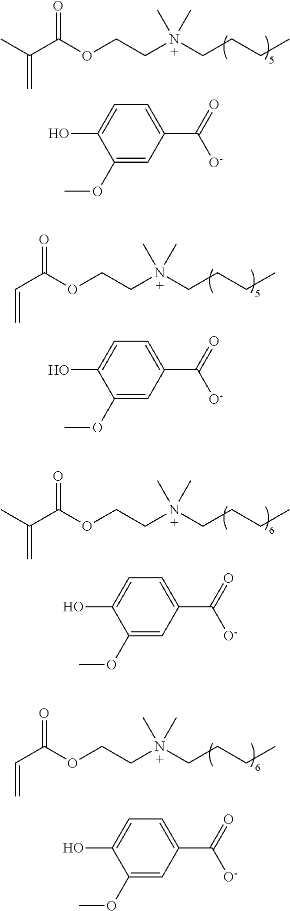

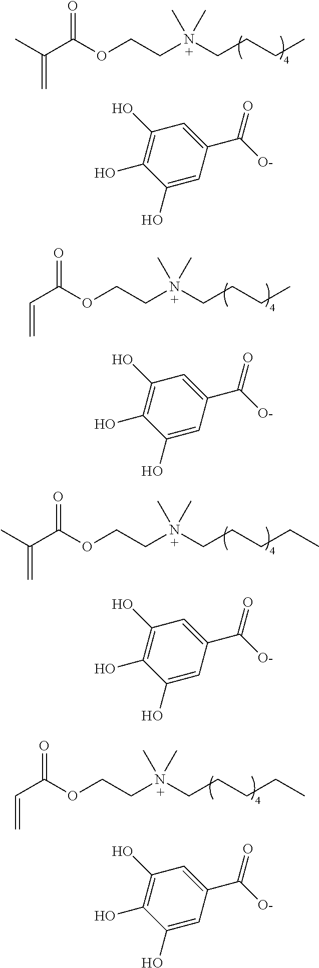

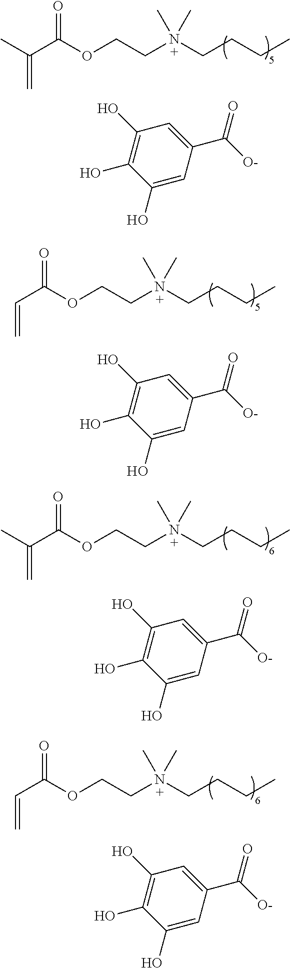

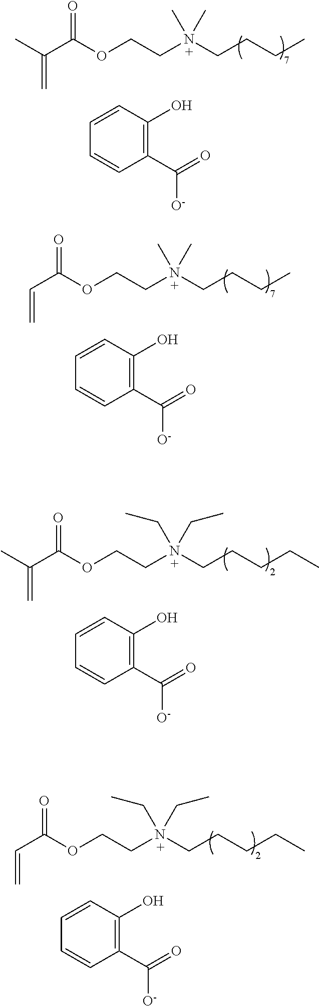

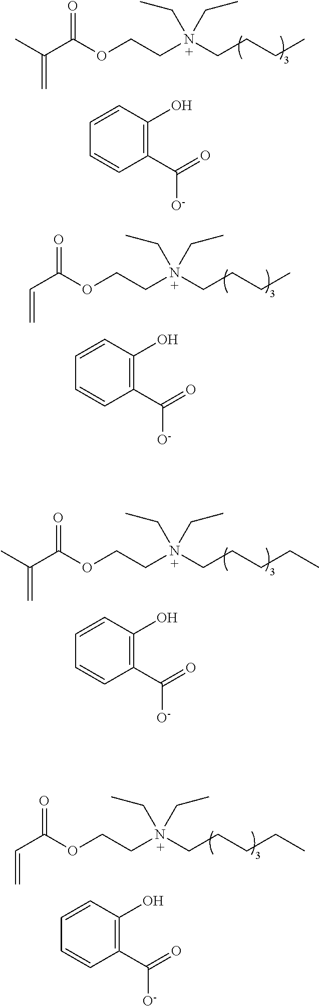

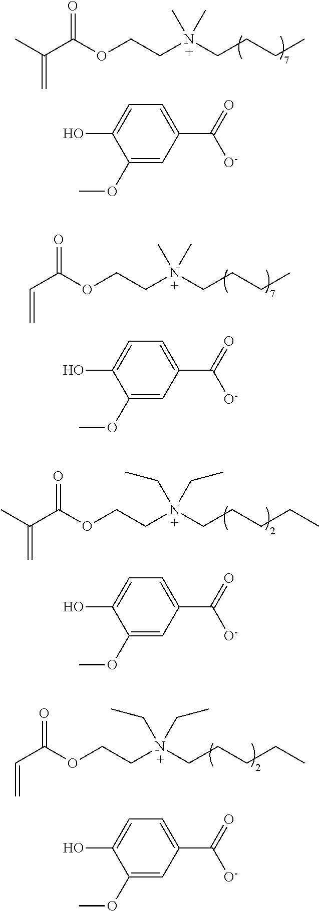

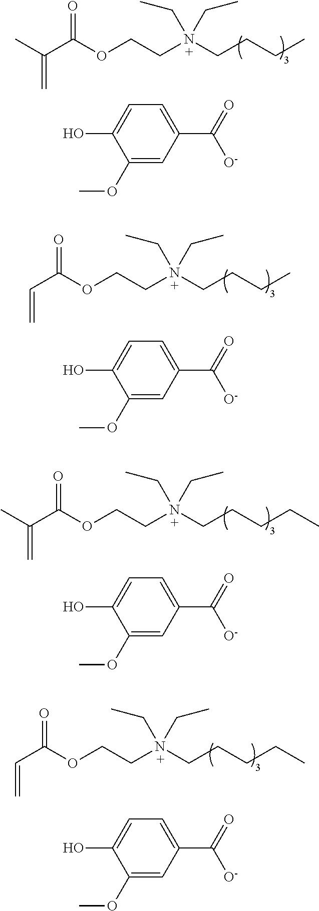

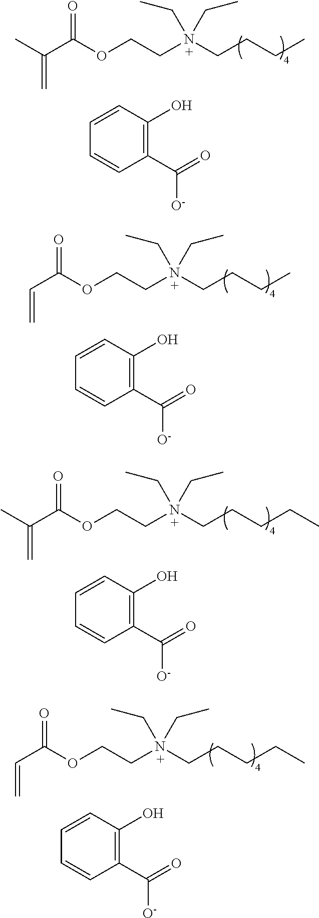

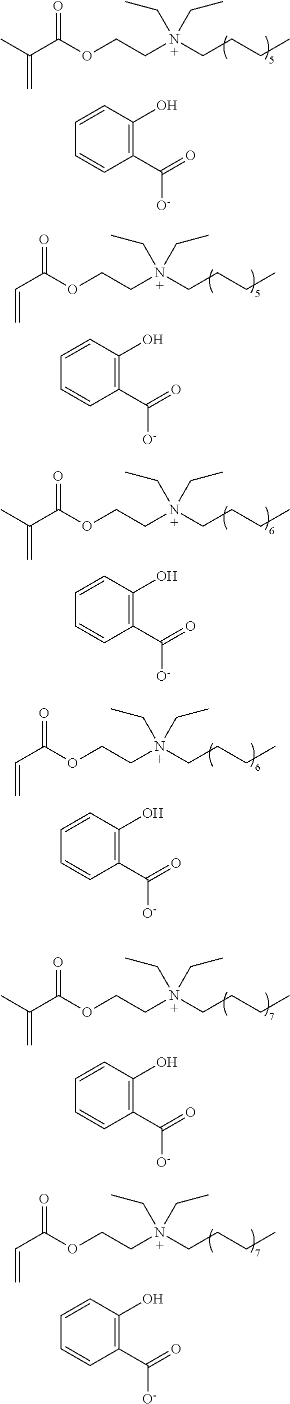

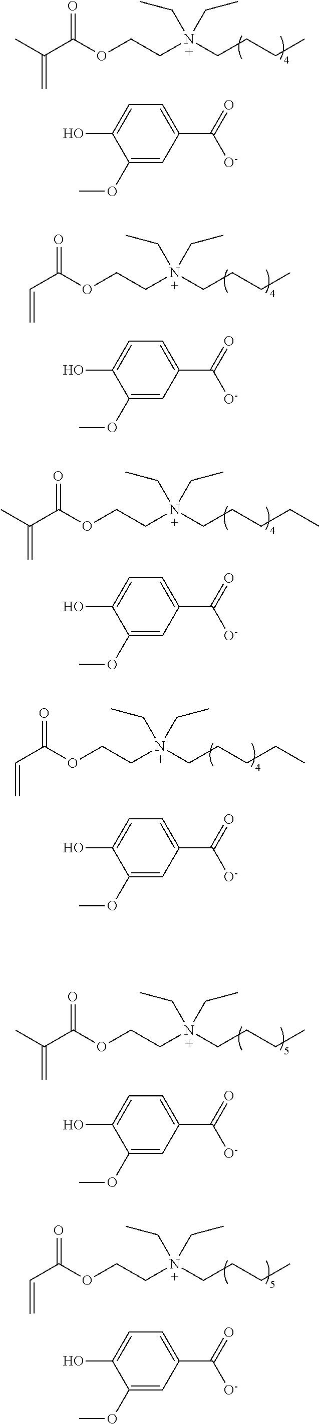

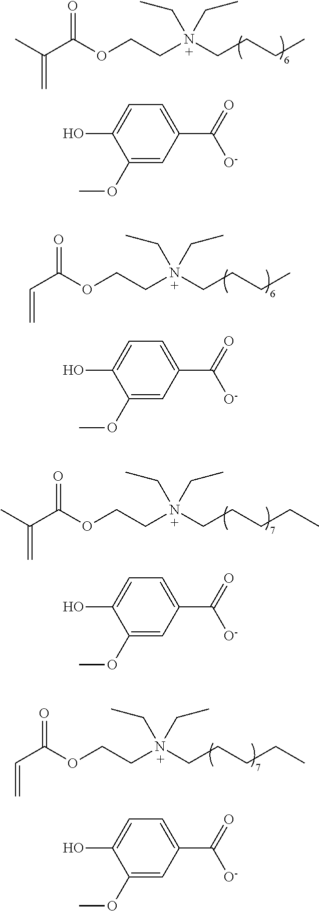

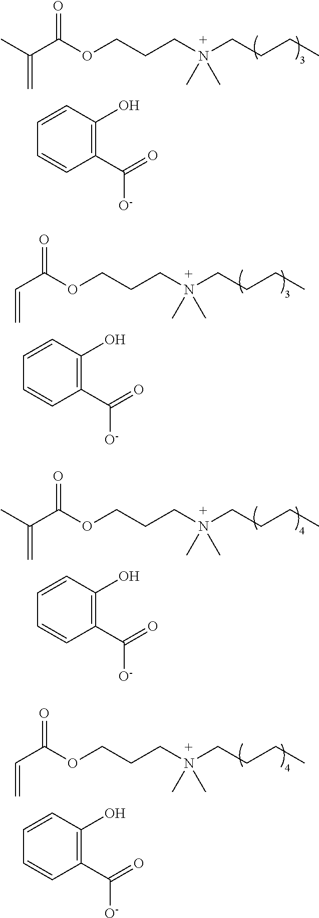

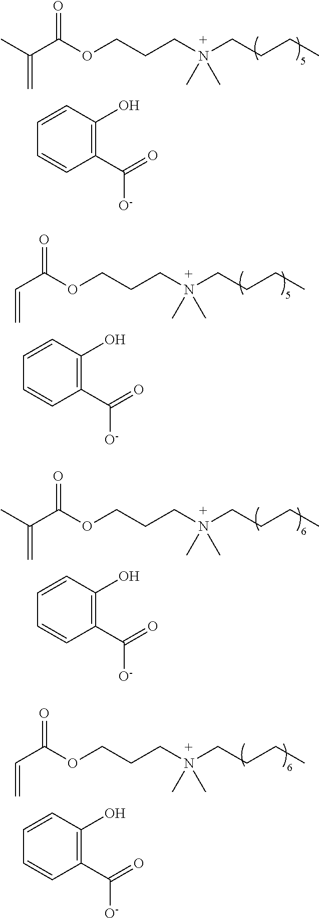

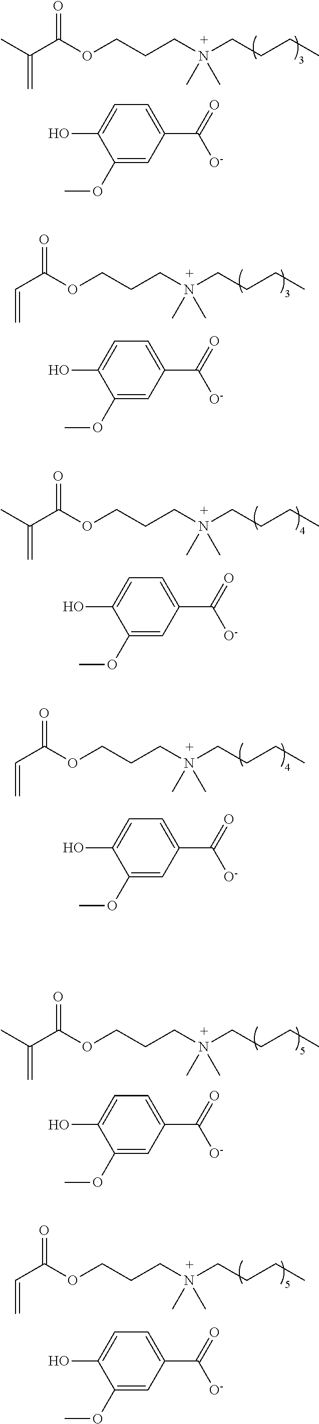

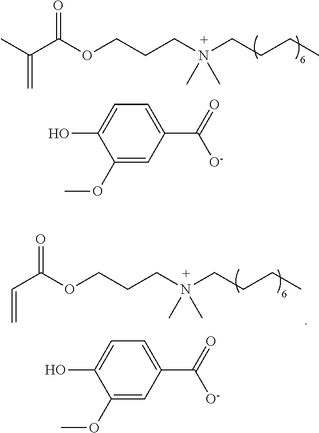

Meanwhile, the compound represented by Chemical Formula 1 may be prepared by a preparation method as in the following Reaction Scheme 1:

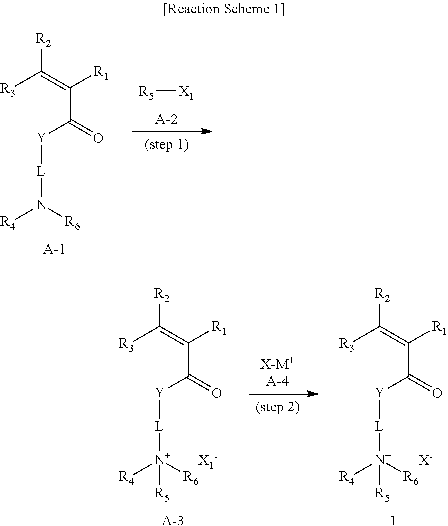

in Reaction Scheme 1, X₁ is halogen, more preferably, bromo or chloro, M is an alkali metal, and descriptions of the remaining substituents are the same as defined in Chemical Formula 1.

Step 1 in Reaction Scheme 1 is a step of preparing a quaternary ammonium salt compound A-3 by reacting a tertiary ammonia compound A-1 with a halide compound A-2, and step 2 is an anion substitution reaction step for substituting the halogen anion of the compound A-3 prepared in step 1 with the desired X⁻ ion. The above preparation method may be more specified in Preparation Examples to be described later.

Further, the compound may exhibit excellent antibacterial effects against microorganisms, particularly, at least one of Gram-positive bacteria or Gram-negative bacteria.

More specifically, the compound may exhibit the antibacterial property against one or more kinds of bacteria, categorized as the Gram-positive bacteria. Alternatively, the compound may exhibit the antibacterial property against one or more kinds of bacteria, categorized as the Gram-negative bacteria. Alternatively, the compound may exhibit the antibacterial property against one or more kinds of bacteria, categorized as the Gram-negative bacteria and one or more kinds of bacteria, categorized as the Gram-positive bacteria.

In this regard, the Gram-negative bacteria against which the compound may exhibit the antibacterial property may be Proteus mirabilis, or Escherichia coli, and the Gram-positive bacteria against which the compound may exhibit the antibacterial property may be Enterococcus faecalis, but are not limited thereto. More preferably, the compound may exhibit the antibacterial property against both of the Gram-positive bacteria and the Gram-negative bacteria. In this regard, the meaning that the compound exhibits the antibacterial property may be confirmed by an antibacterial rate of 50% or more, as measured in an antibacterial property test using absorbance, described later.

Here, Proteus mirabilis is a Gram-negative, rod-shaped, facultatively anaerobic or aerobic bacterium, and distributed in various environments, and may infect the respiratory tract or skin of humans and animals to cause urinary tract-related diseases. In particular, it is known that when a person is infected with the Proteus mirabilis, it may cause urinary tract infection or acute pyelonephritis. In addition, Proteus mirabilis alkalizes urine to allow ammonia to be excreted, which may cause odor.

Specifically, the antibacterial property of the compound against E. coli may be evaluated by measuring absorbance, and an antibacterial rate of the compound against E. coli, as calculated by the following Equation 1, may be 70% or more.

$$Antibacterial rate ( \% ) = ( 1 - {A}_{s} / {A}_{0} ) \times 100 \qquad [ Equation 1 ]$$

in the equation,
Aₛ(Aₛₐₘₚₗₑ) represents absorbance of the culture medium containing the bacteriostatic substance at a wavelength of 600 nm, and
A₀(A_{reference}) represents absorbance of the E. coli pure culture medium without the bacteriostatic substance at a wavelength of 600 nm.

More preferably, the antibacterial rate of the compound against E. coli, as calculated by Equation 1, may be 70% or more, 70.6% or more, 75.5% or more, 80% or more, 90% or more, 95% or more, 95.3% or more, 95.8% or more, 96% or more, 97% or more, 97.3% or more, 98% or more, 98.1% or more, and 100% or less.

Evaluation of the antibacterial property of the compound against Proteus mirabilis and Enterococcus faecalis may also be performed in the same manner as in the evaluation of the antibacterial property of the compound against E. coli.

### Antibacterial Agent

Meanwhile, according to another aspect, provided is an antibacterial agent including the above-described compound represented by Chemical Formula 1. The antibacterial agent may include the compound having the excellent bacterial growth-inhibitory effect as described above, thereby exhibiting excellent antibacterial property.

In this regard, the antibacterial agent may be mixed in an article requiring antibacterial property, or applied or coated onto the article. It may be applied to various household chemical products, in which harmful bacteria grow easily, without limitation, for examples, articles requiring such antibacterial property, such as humidifiers, water tanks, refrigerators, air washers, aquariums, air purifiers, agricultural films, freshness-keeping materials, containers for processed foods, and packaging materials for electronic parts, etc.

Further, the antibacterial agent including the compound may be prepared in various forms, such as an antibacterial coating composition, an antibacterial resin, an antibacterial plastic, etc., depending on the application required. Furthermore, the antibacterial agent may further include other types of resins and/or solvents to facilitate its mixing with or application onto the antibacterial articles.

Hereinafter, the actions and effects of the present invention will be described in more detail with reference to the specific exemplary embodiments of the present invention. However, these exemplary embodiments are provided only for illustrating the present invention, and the scope of the present invention is not defined thereby.

### Preparation Example A: Preparation of Compound A

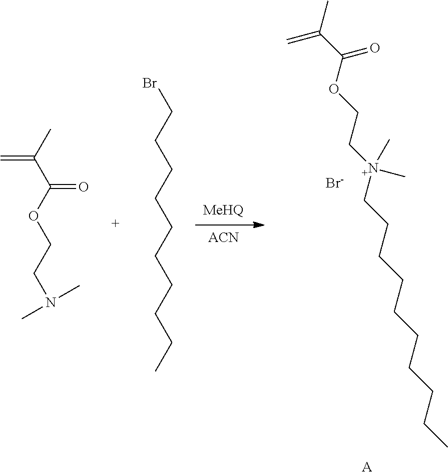

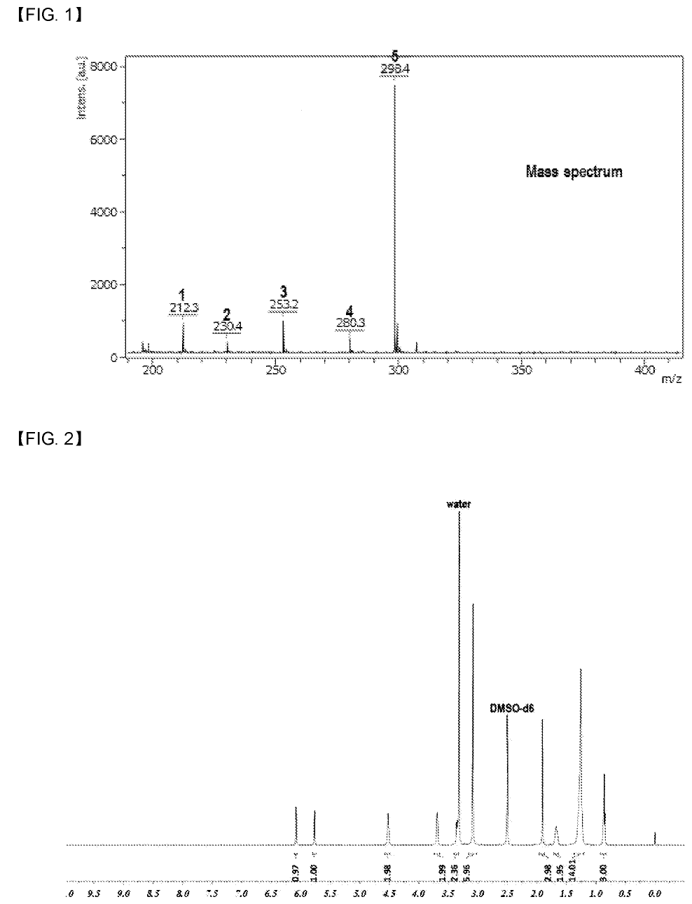

7.86 g of 2-(dimethylamino)ethyl methacrylate and 11.06 g of bromodecane were put in 30 mL of acetonitrile (ACN), followed by stirring. Thereafter, 4 mg of p-methoxyphenol (4-methoxyphenol; MeHQ) was added thereto, and a reflux reaction was allowed at 60° C. for 24 hours. After the reaction was completed, the reaction product was added to 300 mL of diethyl ether, and precipitated by stirring, and filtered to obtain Compound A. As a result of MALDI-TOF mass spectrometry and ¹H NMR analysis of the obtained compound A, a value corresponding to the cation of Compound A was detected. Meanwhile, MS spectrum and ¹H NMR spectrum of Compound A are shown in FIGS. 1 and 2, respectively.

[MS-H]⁺=298

¹H NMR (500 MHZ, DMSO-d₆, δ[ppm]): 6.08(1H), 5.77(1H), 4.52(2H), 3.69(2H), 3.35(2H), 3.09(6H), 1.91(3H), 1.67(2H), 1.25(14H), 0.96(3H)

### Preparation Example B: Preparation of Compound B

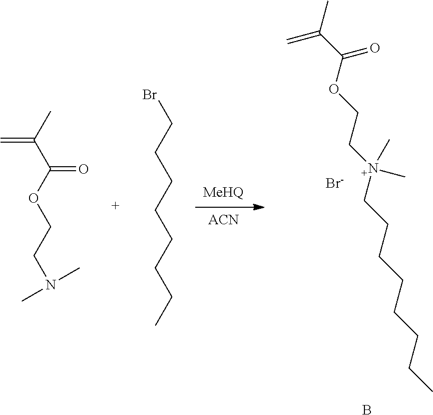

Compound B was synthesized in the same manner as in Preparation Example A, except that bromooctane was used instead of bromodecane in Preparation Example A.

[MS-H]⁻=270

### Preparation Example C: Preparation of Compound C

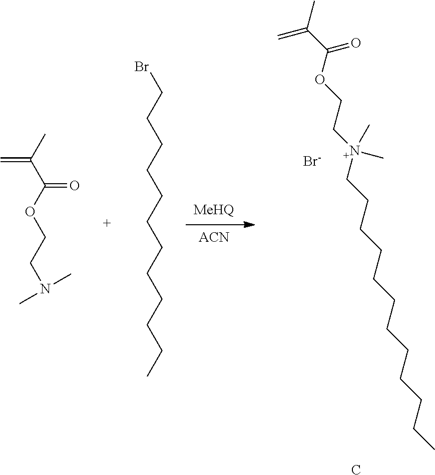

Compound C was synthesized in the same manner as in Preparation Example A, except that bromododecane was used instead of bromodecane in Preparation Example A.

[MS-H]⁺=326

### Preparation Example D: Preparation of Compound D

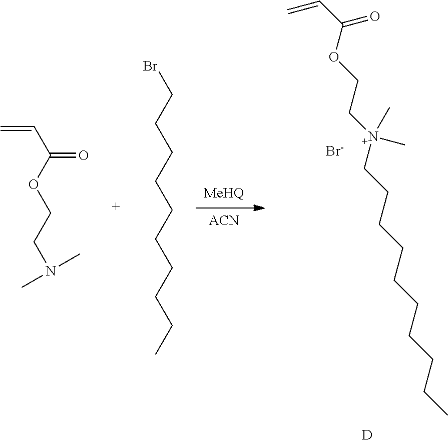

7.16 g of 2-(dimethylamino)ethyl acrylate and 11.06 g of bromodecane were put in 30 mL of acetonitrile (ACN), followed by stirring. Thereafter, 4 mg of p-methoxyphenol (MeHQ) was added thereto, and a reflux reaction was allowed at 60° C. for 24 hours. After the reaction was completed, the reaction product was added to 250 ml of diethyl ether, and precipitated by stirring, and filtered to obtain Compound D. As a result of MALDI-TOF mass spectrometry and ¹H NMR analysis of the obtained compound D, a value corresponding to the cation of Compound D was detected.

[MS-H]⁺=284

1H NMR (500 MHZ, DMSO-d₆, δ[ppm]): 6.48(1H), 6.11(1H), 5.94(1H), 4.67(2H), 4.13(2H), 3.58(2H), 3.48(6H), 1.74(2H), 1.27(14H), 0.86(3H)

### Preparation Example E: Preparation of Compound E

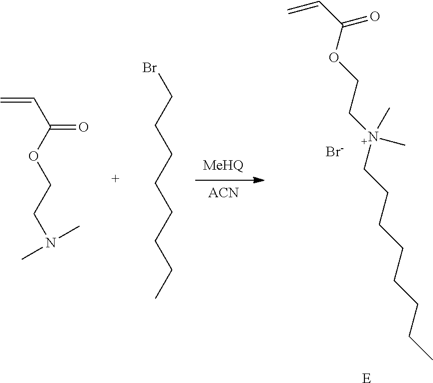

Compound E was synthesized in the same manner as in Preparation Example D, except that bromooctane was used instead of bromodecane in Preparation Example D.

[MS-H]⁺=256

### Preparation Example 1: Preparation of Compound 1

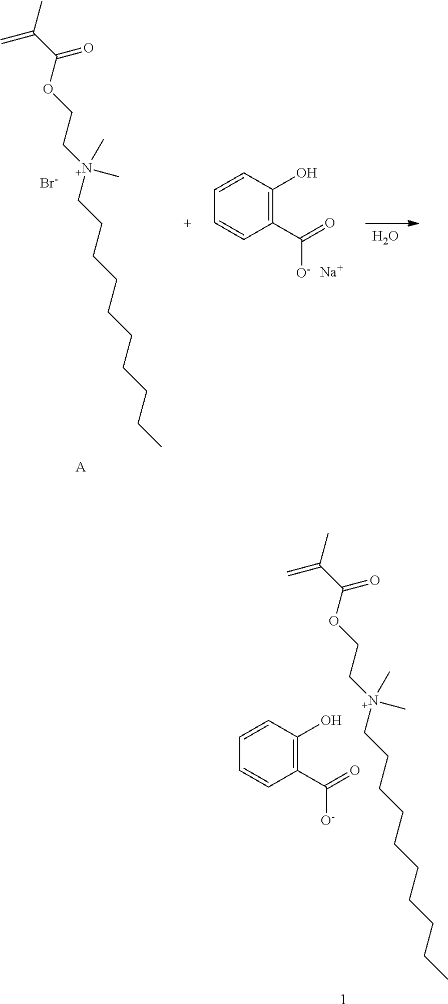

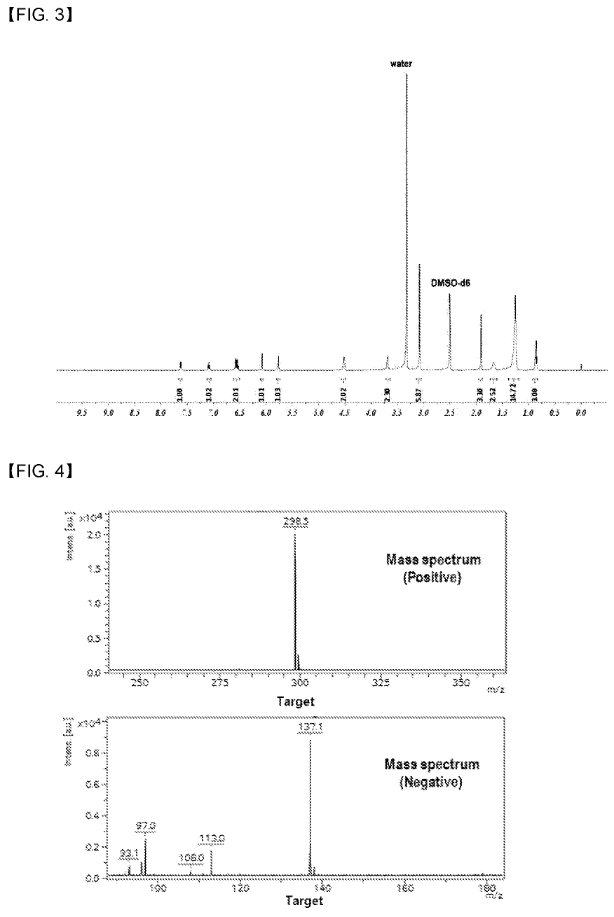

20 g of Compound A synthesized in Preparation Example A was dissolved in 100 mL of DI water, and 11.3 g of sodium salicylate dissolved in 50 ml of water was mixed and stirred at room temperature for 24 hours to perform an anion substitution reaction. Then, an organic layer was extracted from the reaction product with ethyl acetate (EA)/DI water to remove the solvent, and recrystallization was performed with EA to obtain Compound 1. As a result of ¹H NMR analysis, a peak corresponding to the anion of Compound 1 was observed, and as a result of inorganic analysis, Compound 1 showed a remarkably low Br content, as compared to Compound A, indicating that the substitution normally occurred. Meanwhile, ¹H NMR spectrum and mass spectrum of Compound 1 are shown in FIGS. 3 and 4, respectively, and the results of measuring the Br contents of Compound 1 and Compound A using a combustion ion chromatography (C-IC) analyzer are shown in Table 1 below.

[MS-H]⁺=298

[M-H]⁻=137

¹H NMR (500 MHZ, DMSO-d₆, δ[ppm]): 7.63(1H), 7.09(1H), 6.58(2H), 6.08(1H), 5.76(1H), 4.53(2H), 3.69(2H), 3.32(2H), 3.08(6H), 1.91(3H), 1.67(2H), 1.25(14H), 0.86(3H)

TABLE 1

| | Br content (wt %) |
|---|---|
| Compound A | 15.4 |
| Compound 1 | 2.2 |

### Preparation Example 2: Preparation of Compound 2

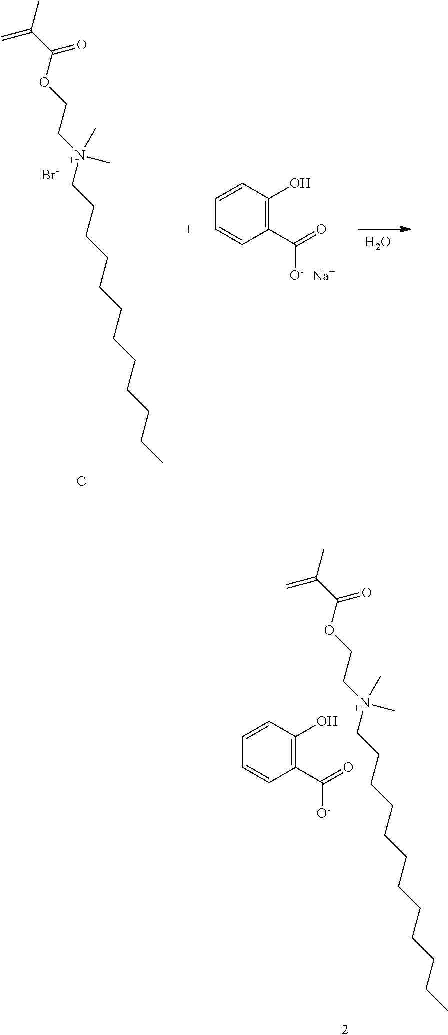

Compound 2 was synthesized in the same manner as in Preparation Example 1, except that Compound C prepared in Preparation Example C was used instead of Compound A in Preparation Example 1.

[MS-H]⁻=326

[M-H]⁻=137

### Preparation Example 3: Preparation of Compound 3

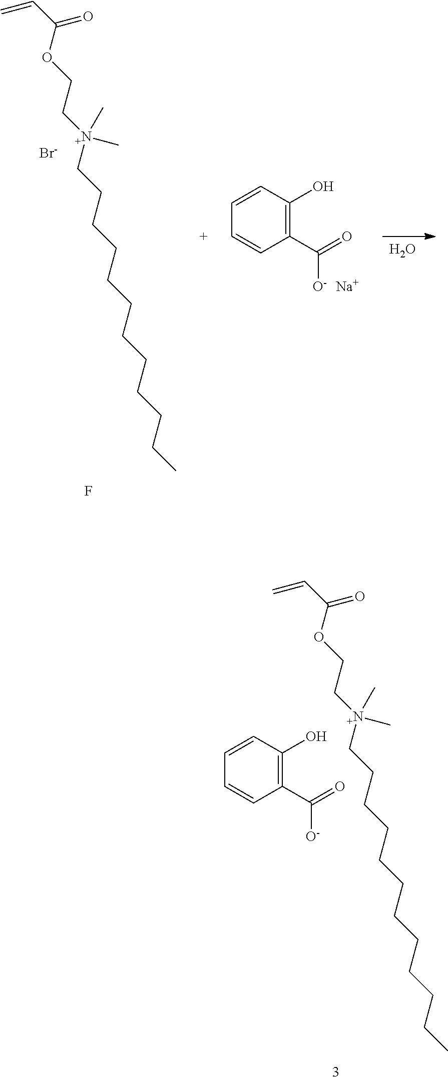

Compound 3 was synthesized in the same manner as in Preparation Example 1, except that compound F (N-(2-(acryloyloxy)ethyl)-N,N-dimethyldodecane-1-aminium bromide) was used instead of Compound A in Preparation Example 1.

[MS-H]⁻=312

[M-H]⁻=137

### Preparation Example 4: Preparation of Compound 4

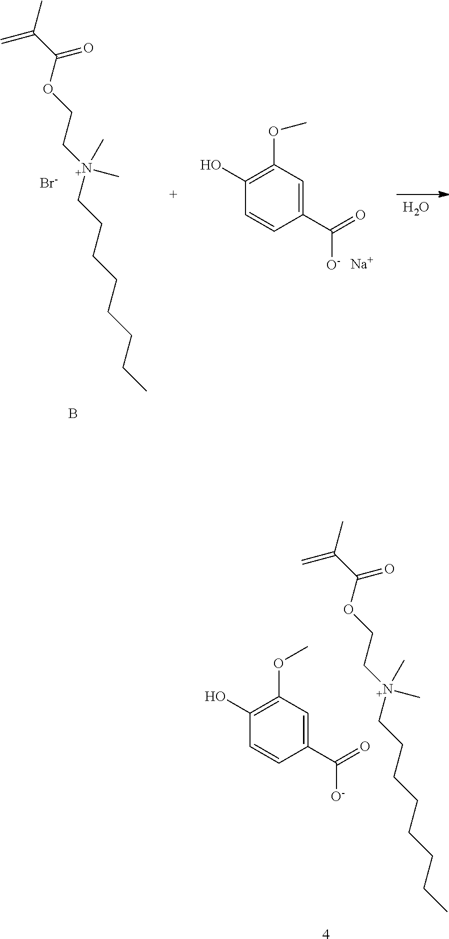

Compound 4 was synthesized in the same manner as in Preparation Example 1, except that compound B prepared in Preparation Example B was used instead of Compound A and sodium vanillate was used instead of sodium salicylate in Preparation Example 1.

[MS-H]⁺=284

[M-H]⁻=167

### Preparation Example 5: Preparation of Compound 5

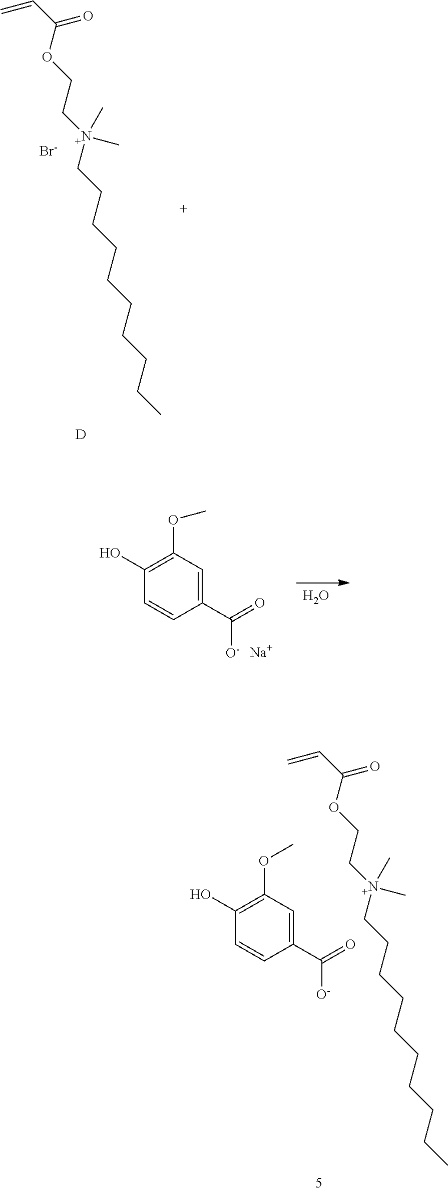

Compound 5 was synthesized in the same manner as in Preparation Example 1, except that Compound D prepared in Preparation Example D was used instead of Compound A and sodium vanillate was used instead of sodium salicylate in Preparation Example 1.

[MS-H]⁺=256

[M-H]⁻=167

### Preparation Example 6: Preparation of Compound 6

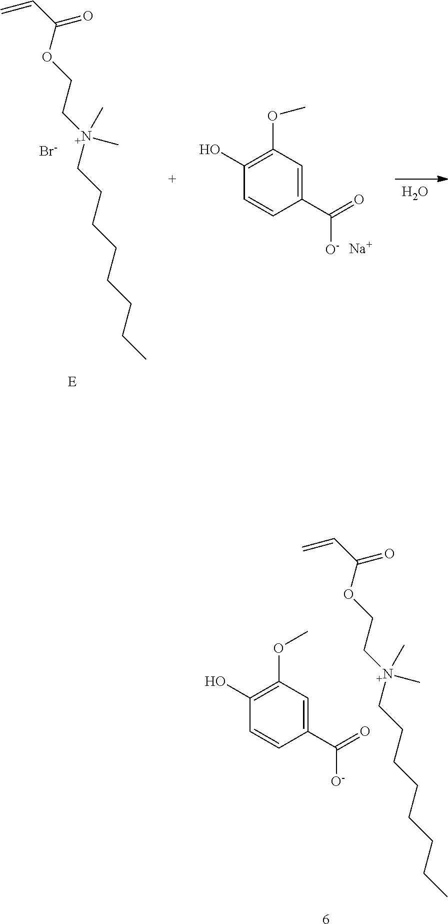

Compound 6 was synthesized in the same manner as in Preparation Example 1, except that Compound E prepared in Preparation Example E was used instead of Compound A and sodium vanillate was used instead of sodium salicylate in Preparation Example 1.

[MS-H]⁺=284

[M-H]⁻=167

### Experimental Example—Evaluation of Antibacterial Property

Unless otherwise indicated, the following property evaluation was performed at constant temperature and constant humidity (23±1° C., relative humidity 50±10% ).

(1) Evaluation of Antibacterial Property Against E. coli

25 mL of a nutrient broth culture medium inoculated with E. coli (ATCC 25922) at 3000 CFU/mL was transferred to a 50 ml conical tube, and then the antibacterial compounds (bacteriostatic substances) prepared in Preparation Examples and Comparative Preparation Examples were injected in each amount described in Table 2 below, followed by sufficiently mixing. Thereafter, incubation was performed for 16 hours in a shaking incubator (Visiontech, VS-37SIF) maintained at 35° C.

The incubated solution was diluted to ⅕ using 1×PBS buffer solution, and absorbance at the wavelength of 600 nm was measured for the diluted solution using a UV-Vis Spectrophotometer (K Lab, Optizen POP). In addition, E. coli (ATCC 25922) was cultured in a pure culture medium without the antibacterial compound for 16 hours in a shaking incubator (VISION TECH, VS-37SIF) maintained at 35° C., which was prepared as a control, and absorbance at the wavelength of 600 nm was measured in the same manner as above.

Then, antibacterial rates (%) of E. coli (ATCC 25922) were calculated according to the following Equation 1, and the results are shown in Table 2 below.

$$Antibacterial rate ( \% ) = ( 1 - {A}_{s} / {A}_{0} ) \times 100 \qquad [ Equation 1 ]$$

in the equation,
Aₛ(Aₛₐₘₚₗₑ) represents absorbance of the culture medium containing the bacteriostatic substance at a wavelength of 600 nm, and
A₀(A_{reference}) represents absorbance of the E. coli pure culture medium without the bacteriostatic substance at a wavelength of 600 nm.

TABLE 2

| | | Injection | Anti- |
|---|---|---|---|
| | Kind of | amount of | bacterial |
| | compound | compound (g) | rate (%) |
| Example 1 | Compound 1 | 0.01 | 95.3 |
| Example 2 | Compound 2 | 0.01 | 98.1 |
| Example 3 | Compound 3 | 0.01 | 97.3 |
| Example 4 | Compound 4 | 0.01 | 75.5 |
| Example 5 | Compound 5 | 0.01 | 95.8 |
| Example 6 | Compound 6 | 0.01 | 70.6 |
| Comparative Example 1 | Compound B | 0.01 | 69.2 |
| Comparative Example 2 | Compound E | 0.01 | 62.3 |

Referring to Table 2, it can be seen that the compounds of Examples, each having the conjugate base of aromatic acid, exhibited the improved antibacterial property against E. coli, as compared to the compounds of Comparative Examples, each having the halogen anion. In particular, when comparing (Compound 4 of Example 4 and Compound B of Comparative Example 1) and (Compound 6 of Example 6 and Compound E of Comparative Example 2), respectively, the antibacterial rates of the compounds of Examples, each having the conjugate base of aromatic acid as an anion, were found to be significantly improved, even though they have the same cation structure.

Accordingly, it can be seen that the compound represented by Chemical Formula 1, in which the quaternary ammonium cation and the anion of the conjugate base of aromatic acid are bound, exhibits the excellent antibacterial property against at least one of Gram-positive bacteria or Gram-negative bacteria.

1. A compound represented by Chemical Formula 1: 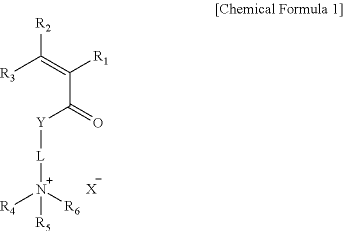
in Chemical Formula b 1,
Y is O, S, or N(R₇),
L is a single bond, an alkylene having 1 to 10 carbon atoms, or an arylene having 6 to 60 carbon atoms,
R₁ to R₃ are each independently hydrogen or methyl,
R₄ to R₇ are each independently hydrogen, a substituted or unsubstituted alkyl having 1 to 20 carbon atoms, or a substituted or unsubstituted aryl having 6 to 60 carbon atoms, and
X⁻ is a conjugate base of an aromatic acid having 6 to 20 carbon atoms, which is substituted with one or more hydroxyl groups,
wherein the conjugate base of the aromatic acid is optionally further substituted with one or more substituents selected from the group consisting of a halogen, an alkyl having 1 to 4 carbon atoms, a haloalkyl having 1 to 4 carbon atoms, and an alkoxy having 1 to 4 carbon atoms.

2. The compound of claim 1,
wherein Y is O.

3. The compound of claim 1,
wherein L is methylene, ethylene, or propylene.

4. The compound of claim 1,
wherein R₁ is hydrogen or methyl, and R₂ and R₃ are hydrogen.

5. The compound of claim 1,
wherein one of R₄ to R₆ is an alkyl having 5 to 20 carbon atoms, and the others are each independently an alkyl having 1 to 4 carbon atoms.

6. The compound of claim 5,
wherein one of R₄ to R₆ is an alkyl having 6 to 16 carbon atoms, and the others are each independently methyl or ethyl.

7. The compound of claim 1,
wherein X⁻ is represented by Chemical Formula 2: 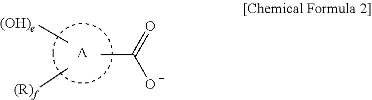
in Chemical Formula 2,
A is a benzene ring, or a naphthalene ring,
R is fluoro, bromo, chloro, iodo, methyl, ethyl, propyl, isopropyl, butyl, isobutyl, sec-butyl, tert-butyl, trifluoromethyl, methoxy, or ethoxy,
e is 1, 2, or 3, and
f is an integer of 0 to 5,
wherein when f is 2 or more, two or more R's are the same as or different from each other.

8. The compound of claim 1,
wherein X⁻ is represented by any one of Chemical Formulae 2-1 to 2-6: 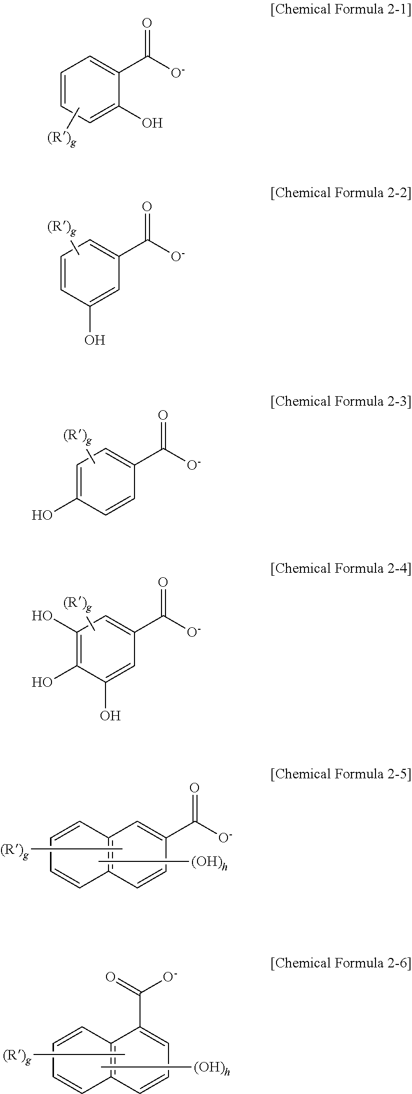
in Chemical Formulae 2-1 to 2-6,
R′ is fluoro, bromo, chloro, methyl, trifluoromethyl, or methoxy,
g is 0, 1, or 2, and
wherein when g is 2, two R's are the same as or different from each other,
h is 1 or 2.

9. The compound of claim 1,
wherein X⁻ is any one selected from the group consisting of:

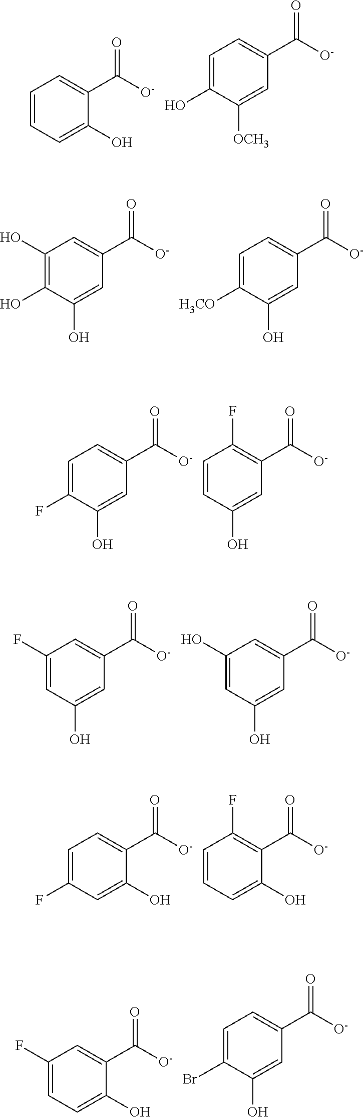

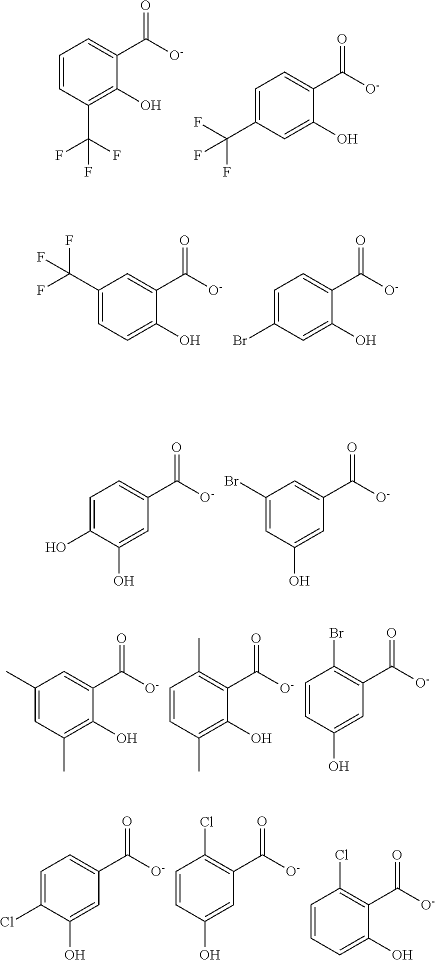

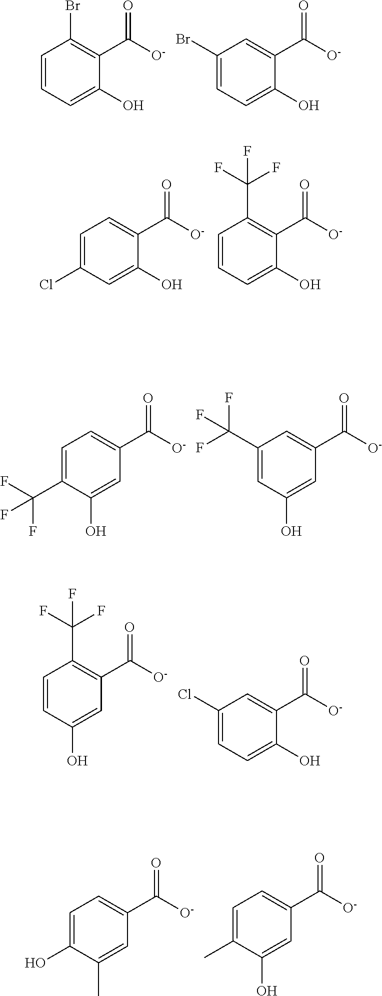

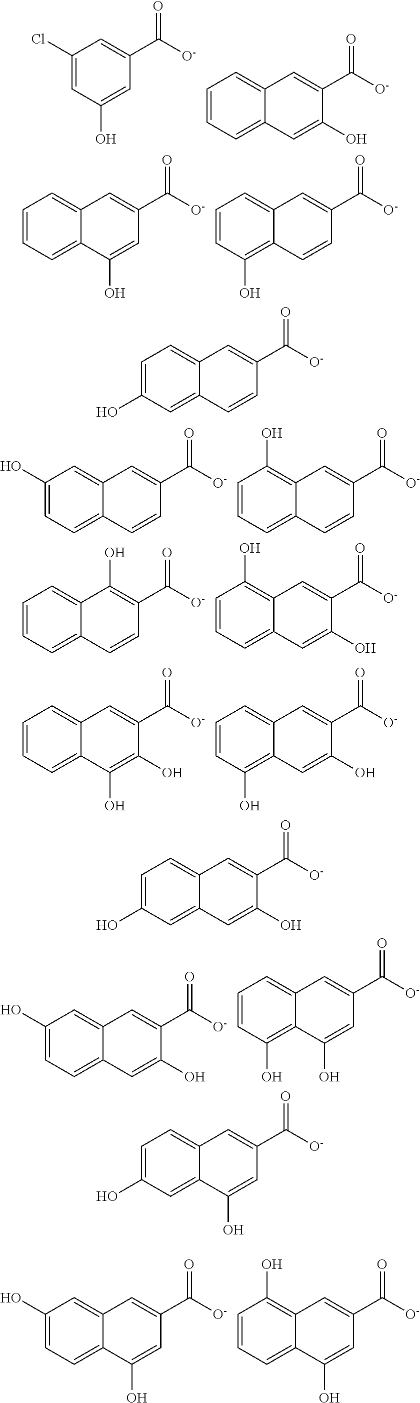

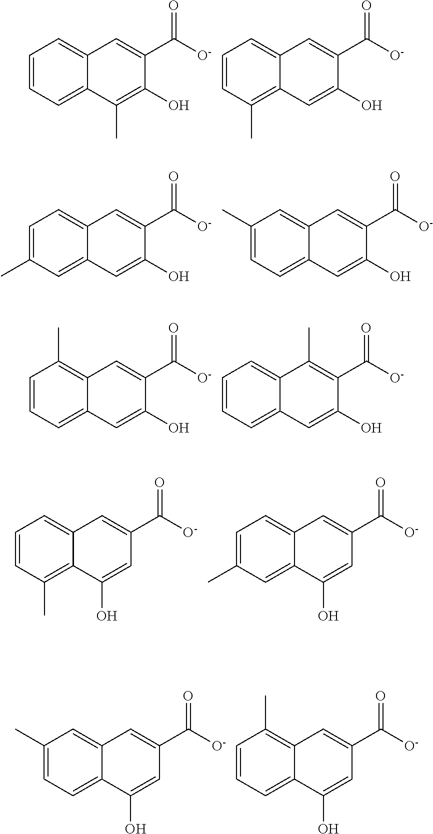

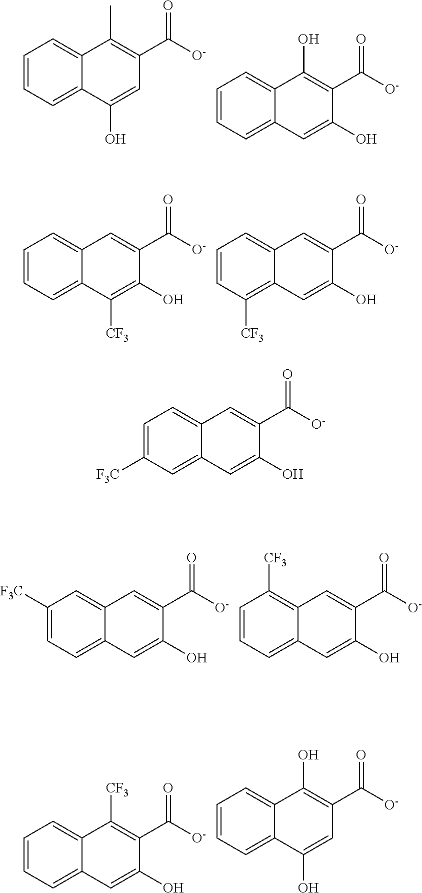

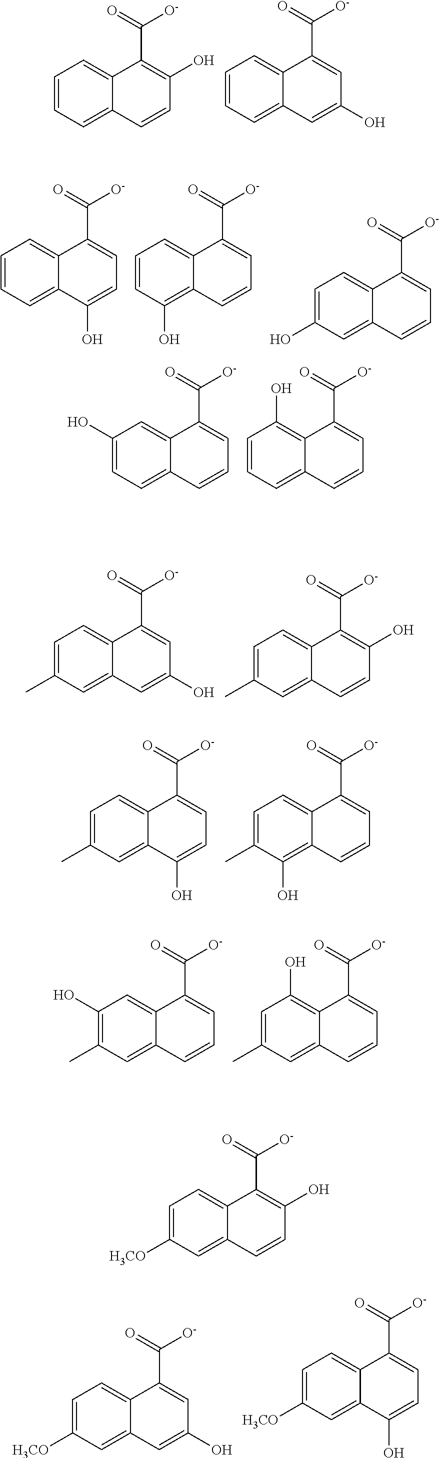

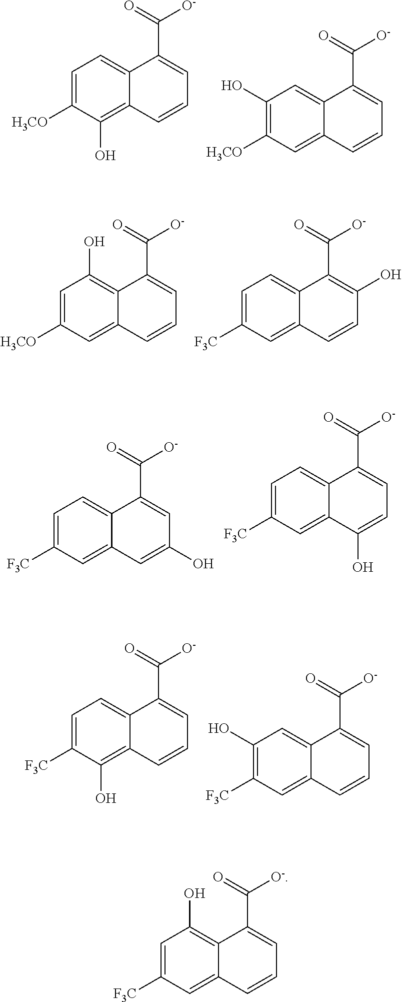

10. The compound of claim 1,
wherein the compound represented by Chemical Formula 1 is represented by any one of the following Chemical Formulae 1-1 to 1-4: 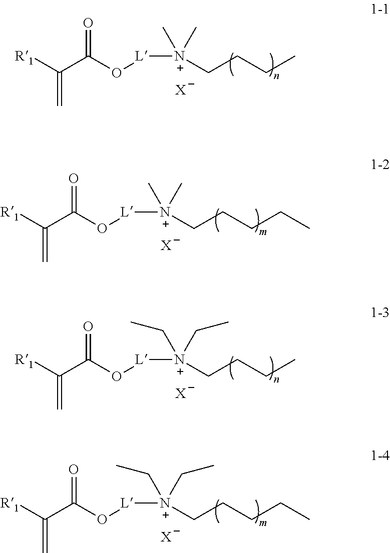
in Chemical Formulae 1-1 to 1-4,
R′₁ is hydrogen or methyl,
L′ is methylene, or ethylene,
X⁻ is the same as defined Chemical Formula 1,
n is an integer of 2 to 7, and
m is an integer of 2 to 6.

11. The compound of claim 1,
wherein the compound is any one selected from the group consisting of the following compounds:

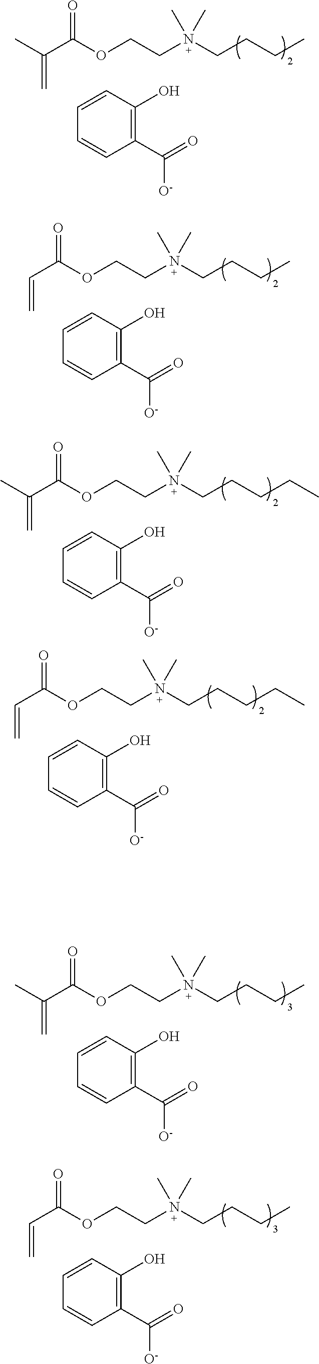

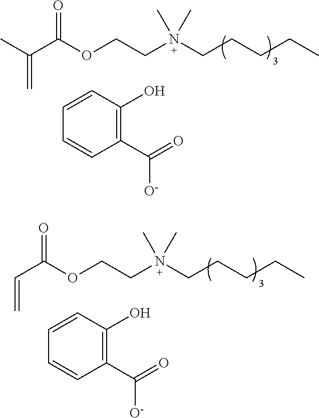

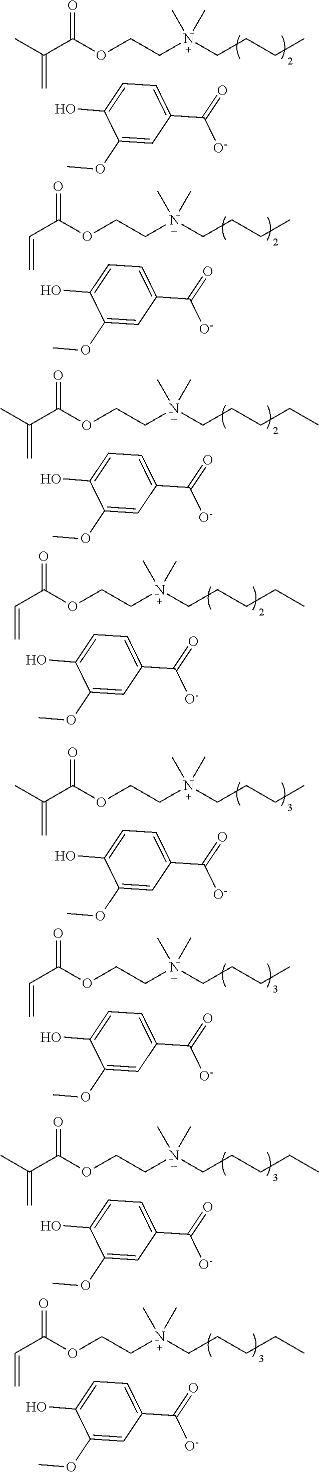

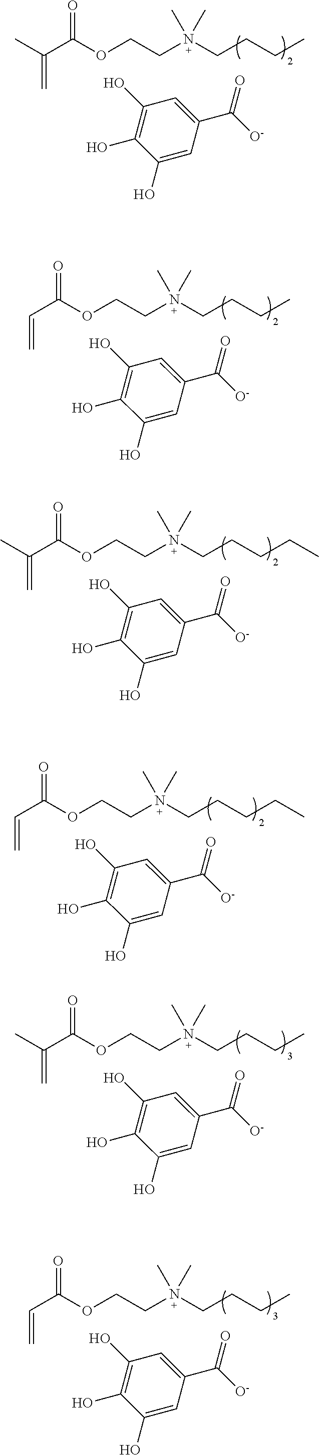

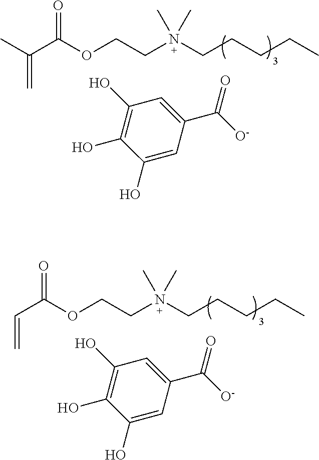

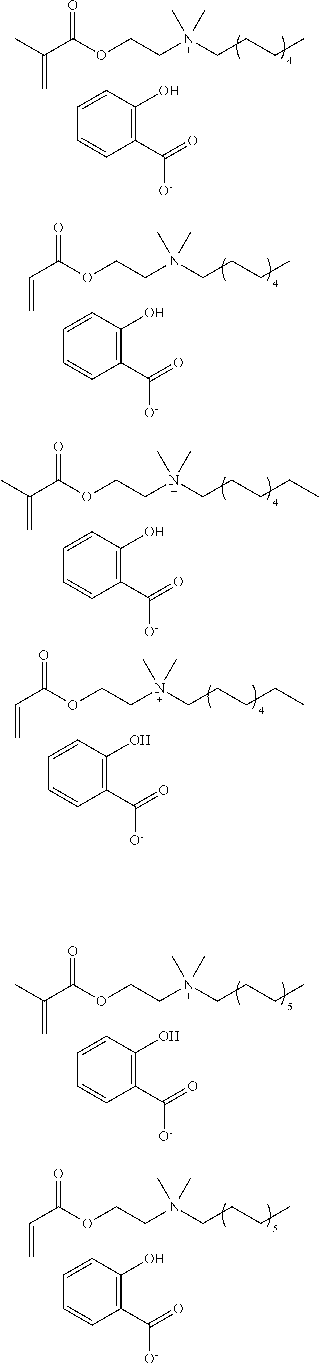

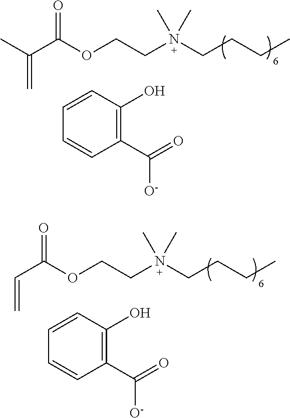

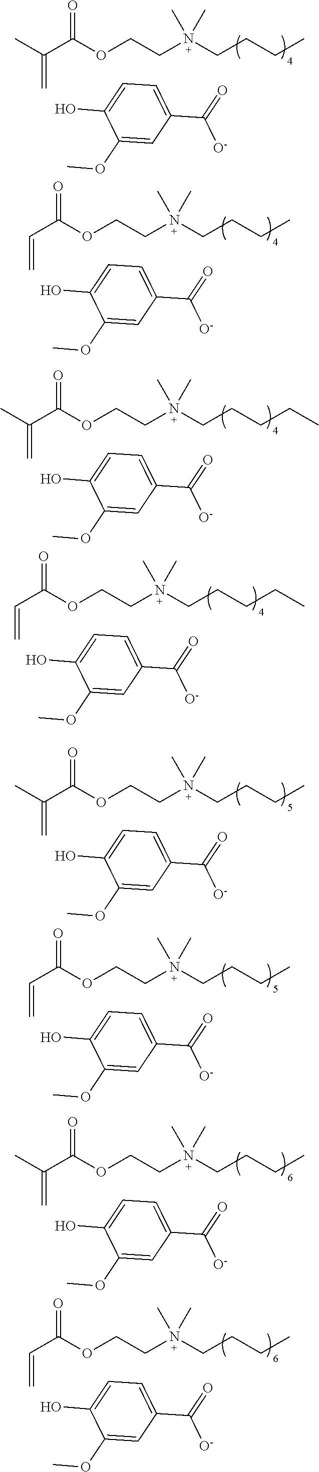

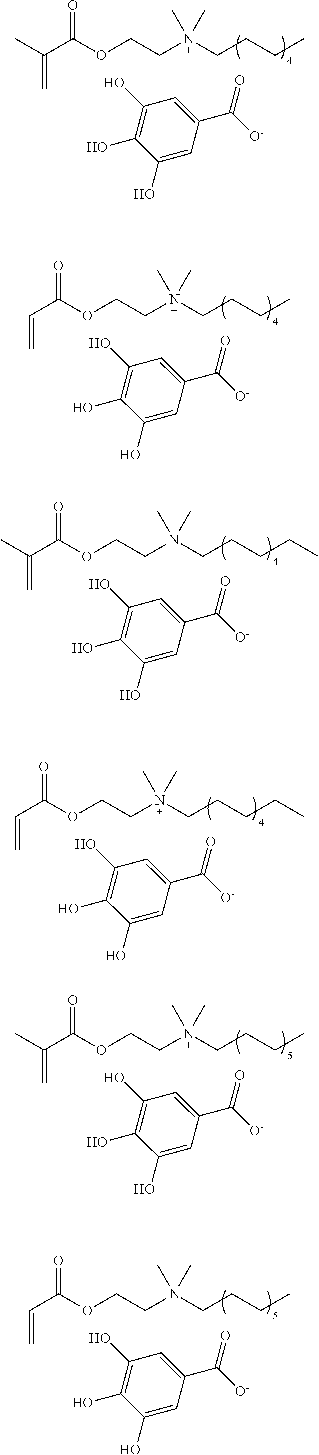

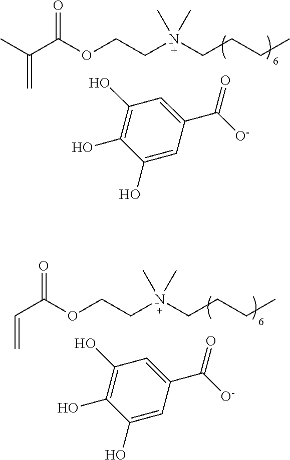

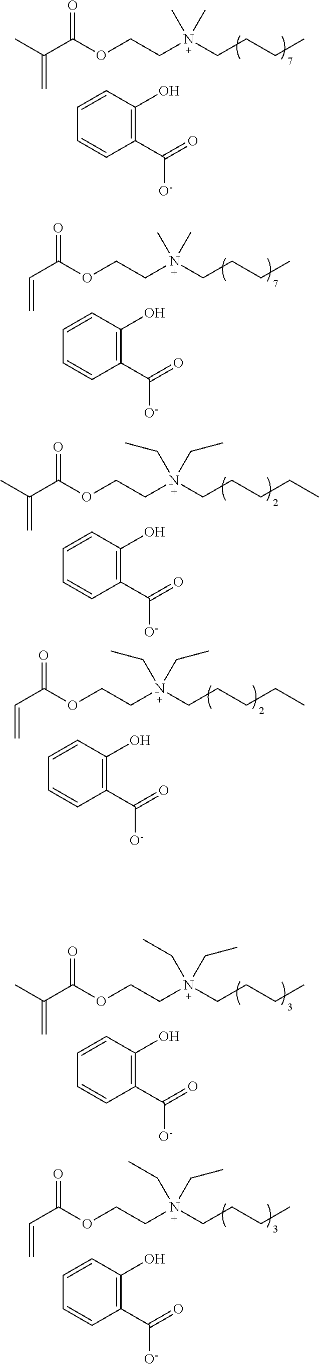

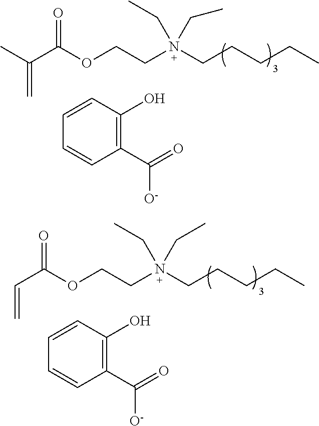

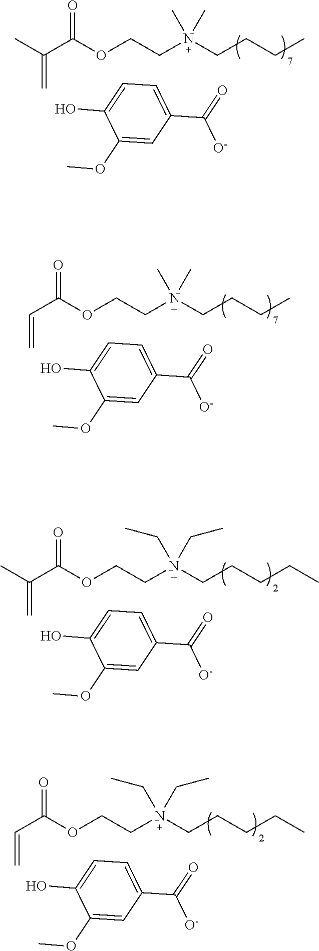

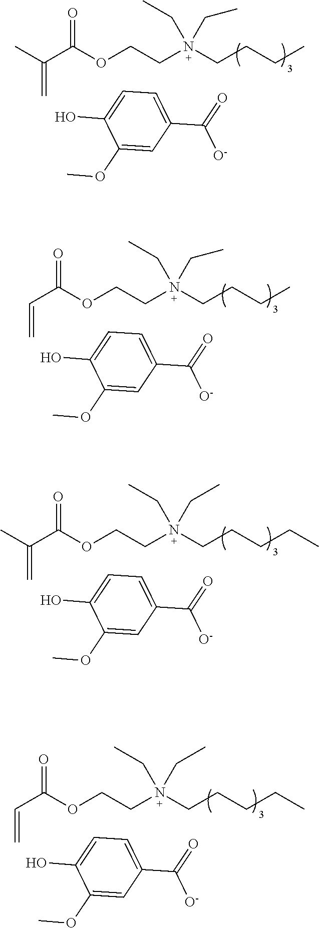

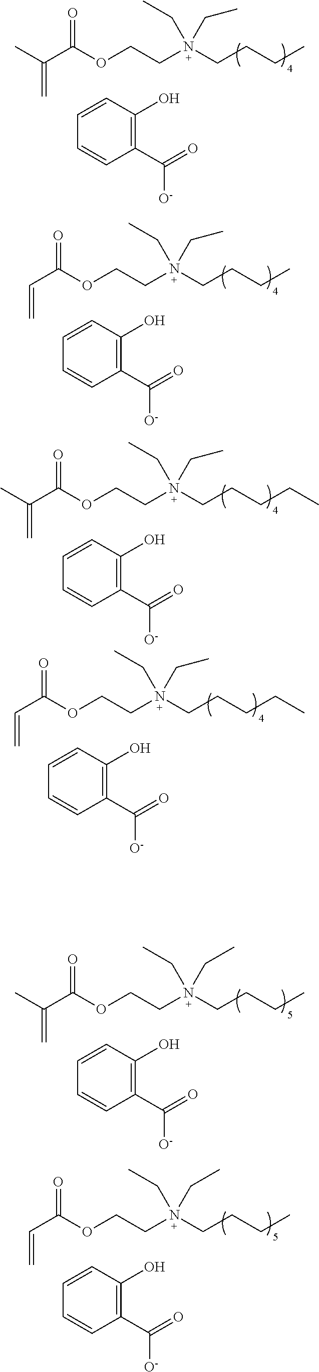

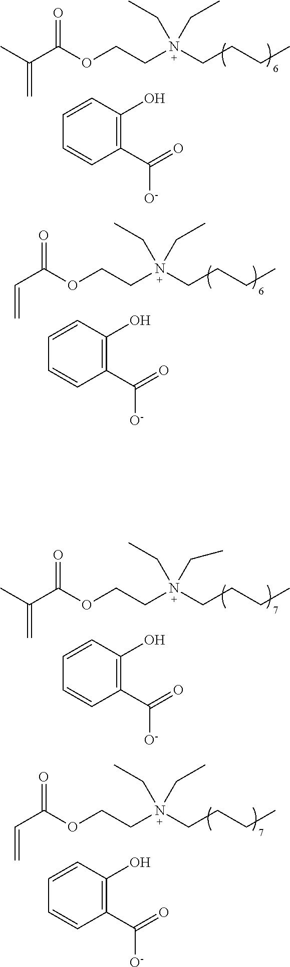

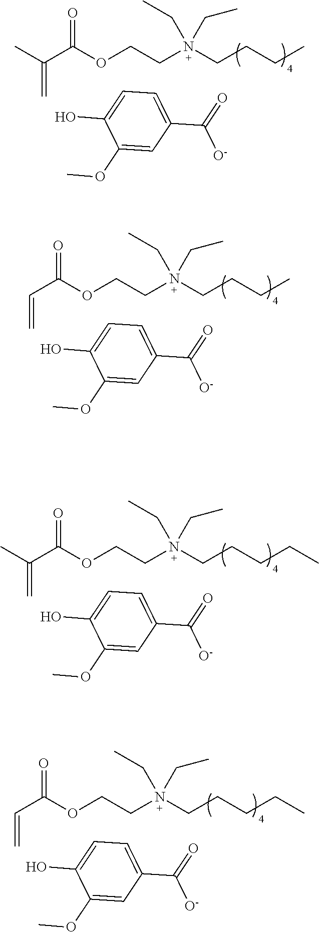

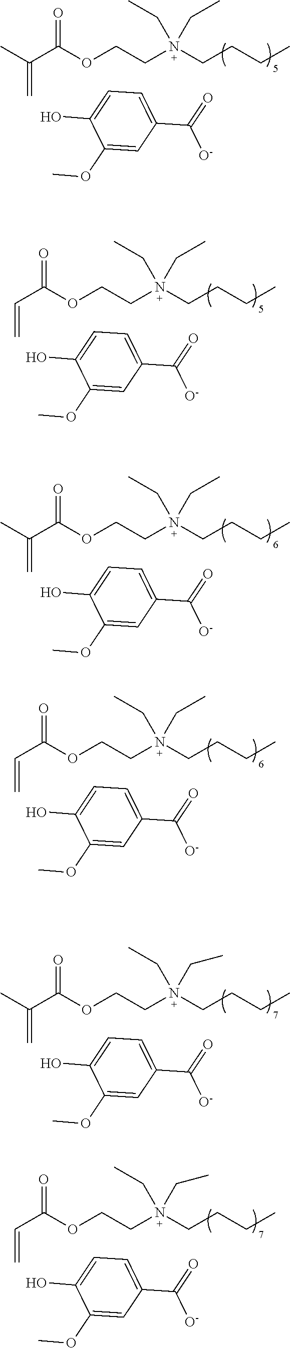

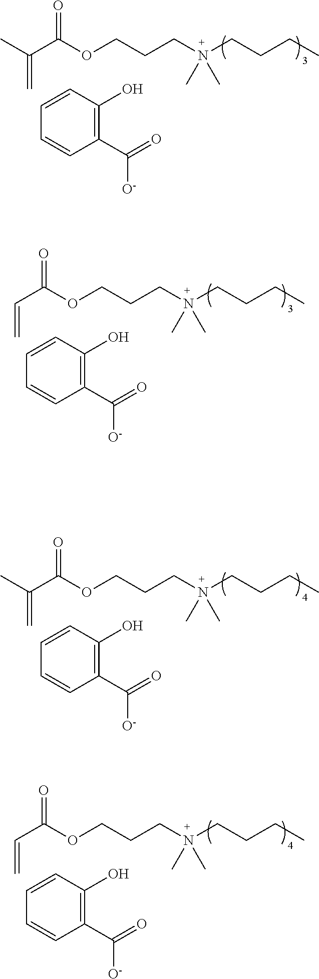

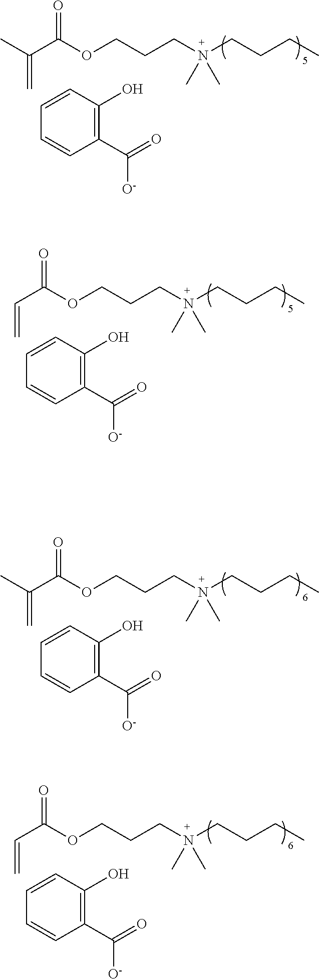

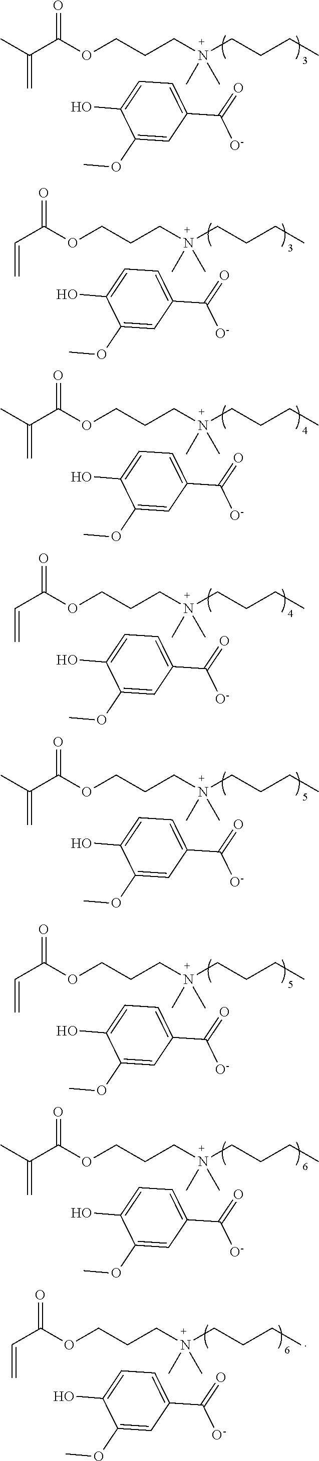

12. The compound of claim 1,
which exhibits an antibacterial property against one or more of a Gram-positive bacterium or a Gram-negative bacterium.

13. The compound of claim 12,
wherein the Gram-negative bacterium is Proteus mirabilis or Escherichia coli, and
the Gram-positive bacterium is Enterococcus faecalis.

14. The compound of claim 12,
which exhibits the antibacterial property against both of Gram-positive bacterium and Gram-negative bacterium.

15. An antibacterial agent comprising the compound of claim 1.

16. The compound of claim 7,
wherein R is fluoro, bromo, chloro, methyl, trifluoromethyl, or methoxy,
e is 1, 2, or 3, and
f is 0, 1, 2, or 3, and
e+f is 1, 2, or 3.
